(19) United States
(12) Patent Application Publication
(10) Pub. No.: US 2016/0296674 A1
(43) Pub. Date: Oct. 13, 2016

(54) NUCLEIC ACID BASED CARDIOVASCULAR THERAPEUTICS

(71) Applicant: Gabor Rubanyi, San Diego, CA (US)

(72) Inventor: Gabor Rubanyi, San Diego, CA (US)

(21) Appl. No.: 14/683,065

(22) Filed: Apr. 9, 2015

(51) Int. Cl. A61L 29/16; A61M 25/10; A61B 17/12; A61L 29/04

(52) U.S. Cl. CPC A61L 29/16; A61L 29/043; A61M 25/10; A61B 17/12136; A61L 2300/204; A61L 2300/258; A61L 2300/414; A61M 2025/105

(57) ABSTRACT

The present invention relates to methods and compositions for delivering a nucleic acid to be expressed in the myocardium of a patient. More specifically, the present invention relates to techniques and polynucleotide constructs for treating heart disease by in vivo delivery of angiogenic transgenes.

### CROSS REFERENCES AND RELATED APPLICATIONS

This application claims the benefit of U.S. Provisional Application 61/505,517 filed Jul. 7, 2011.

### FIELD OF THE INVENTION

The present invention relates to methods and compositions for delivering a nucleic acid to be expressed in the myocardium of a patient. More specifically, the present invention relates to techniques and polynucleotide constructs for treating heart disease by in vivo delivery of angiogenic transgenes.

### BACKGROUND OF THE INVENTION

It has been reported by the American Heart Association (2008 Statistical Supplement), that about 80 million adults in the United States suffer from cardiovascular disease.

Cardiovascular diseases are responsible for almost a million deaths annually in the United States representing over 40% of all deaths. Each year, in the United States, there are about 500,000 new cases of angina pectoris, a common condition of coronary artery disease characterized by transient periods of myocardial ischemia resulting in chest pain. Similarly, each year, more than 400,000 patients are diagnosed with congestive heart failure “CHF,” another manifestation of heart disease that represents the most frequent non-elective cause of hospitalization in the U. S.

Myocardial ischemia is an aspect of heart dysfunction that occurs when the heart muscle (the myocardium) does not receive adequate blood supply and is thus deprived of necessary levels of oxygen and nutrients. Myocardial ischemia may result in a variety of heart diseases including, for example, angina, heart attack and/or congestive heart failure. The most common cause of myocardial ischemia is atherosclerosis (also referred to as coronary artery disease or “CAD”), which causes blockages in the coronary arteries, blood vessels that provide blood flow to the heart muscle. Present treatments for myocardial ischemia include pharmacological therapies, coronary artery bypass surgery and percutaneous revascularization using techniques such as balloon angioplasty. Standard pharmacological therapy is predicated on strategies that involve either increasing blood supply to the heart muscle or decreasing the demand of the heart muscle for oxygen and nutrients. For example, increased blood supply to the myocardium can be achieved by agents such as calcium channel blockers or nitroglycerin.

These agents are thought to increase the diameter of diseased arteries by causing relaxation of the smooth muscle in the arterial walls. Decreased demand of the heart muscle for oxygen and nutrients can be accomplished either by agents that decrease the hemodynamic load on the heart, such as arterial vasodilators, or those that decrease the contractile response of the heart to a given hemodynamic load, such as beta-adrenergic receptor antagonists. Surgical treatment of ischemic heart disease is generally based on the bypass of diseased arterial segments with strategically placed bypass grafts (usually saphenous vein or internal mammary artery grafts). Percutaneous revascularization is generally based on the use of catheters to reduce the narrowing in diseased coronary arteries. All of these strategies are used to decrease the number of, or to eradicate, ischemic episodes, but all have various limitations, some of which are discussed below.

Many patients with heart disease, including many of those whose severe myocardial ischemia resulted in a heart attack, ultimately develop congestive heart failure. Congestive heart failure is defined as abnormal heart function resulting in inadequate cardiac output to meet metabolic needs (Braunwald, E. (ed), In: Heart Disease, W. B. Saunders, Philadelphia, page 426, 1988). An estimated 5 million people in the United States suffer from congestive heart failure. Once symptoms of CHF are moderately severe, the prognosis is worse than most cancers in that only half of such patients are expected to survive for more than 2 years (Braunwald, E. (ed), In: Heart Disease, W. B. Saunders, Philadelphia, page 471-485, 1988). Medical therapy can initially attenuate the symptoms of CHF (e.g., edema, exercise intolerance and breathlessness), and in some cases prolong life. However, the prognosis for this disease, even with medical treatment, remains grim, and the incidence of CHF has been increasing (see, e.g., Baughman, K., Cardiology Clinics 13: 27-34, 1995).

Symptoms of CHF include breathlessness, fatigue, weakness, leg swelling and exercise intolerance. On physical examination, patients with heart failure tend to have elevations in heart and respiratory rates, rales (an indication of fluid in the lungs), edema, jugular venous distension, and, in general, enlarged hearts. The most common cause of CHF is atherosclerosis which, as discussed above, causes blockages in the coronary arteries that supply blood to the heart muscle. Thus, congestive heart failure is most commonly associated with coronary artery disease that is so severe in scope or abruptness that it results in the development of chronic or acute heart failure. In such patients, extensive and/or abrupt occlusion of one or more coronary arteries precludes adequate blood flow to the myocardium, resulting in severe ischemia and, in some cases, myocardial infarction or death of heart muscle. The consequent myocardial necrosis tends to be followed by progressive chronic heart failure or an acute low output state-both of which are associated with high mortality.

Most patients with congestive heart failure tend to develop enlarged, poorly contracting hearts, a condition referred to as “dilated cardiomyopathy” (or DCM, as used herein). DCM is a condition of the heart typically diagnosed by the finding of a dilated, hypocontractile left and/or right ventricle. Again, in the majority of cases, the congestive heart failure associated with a dilated heart is the result of coronary artery disease, often so severe that it has caused one or more myocardial infarcts. In a significant minority of cases, however, DCM can occur in the absence of characteristics of coronary artery disease (e.g., atherosclerosis). In a number of cases in which the dilated cardiomyopathy is not associated with CAD, the cause of DCM is known or suspected. Examples include familial cardiomyopathy (such as that associated with progressive muscular dystrophy, myotonic muscular dystrophy, Freidrich's ataxia, and hereditary dilated cardiomyopathy), infections resulting in myocardial inflammation (such as infections by various viruses, bacteria and other parasites), noninfectious inflammations (such as those due to autoimmune diseases, peripartum cardiomyopathy, hypersensitivity reactions or transplantation rejections), metabolic disturbances causing myocarditis (including nutritional, endocrinologic and electrolyte abnormalities) and exposure to toxic agents causing myocarditis (including alcohol, as well as certain chemotherapeutic drugs and catecholamines). In the majority of non-CAD DCM cases, however, the cause of disease remains unknown and the condition is thus referred to as “idiopathic dilated cardiomyopathy” (or “IDCM”). Despite the potential differences in underlying causation, most patients with severe CHF have enlarged, thin-walled hearts (i. e., DCM) and most of those patients exhibit myocardial ischemia (even though some of them may not have apparent atherosclerosis). Furthermore, patients with DCM can experience angina pectoris even though they may not have severe coronary artery disease.

The occurrence of CHF poses several major therapeutic concerns, including progressive myocardial injury, hemodynamic inefficiencies associated with the dilated heart, the threat of systemic emboli, and the risk of ventricular arrhythmias. Traditional revascularization is not an option for treatment of non-CAD DCM, because occlusive coronary disease is not the primary problem. Even for those patients for which the cause of DCM is known or suspected, the damage is typically not readily reversible. For example, in the case of adriamycin-induced cardiotoxicity, the cardiomyopathy is generally irreversible and results in death in over 60% of afflicted patients. For some patients with DCM, the cause itself is unknown. As a result, there are no generally applied treatments for DCM. Physicians have traditionally focused on alleviating the symptoms presented in a patient exhibiting DCM (e.g., by relieving fluid retention with diuretics, and/or reducing the demand of the heart muscle for oxygen and nutrients with angiotensin converting enzyme inhibitors). As a result, approximately 50% of the patients exhibiting DCM die within two years of diagnosis, often from sudden cardiac arrest associated with ventricular arrhythmias. “Ventricular remodeling” is an aspect of heart disease that often occurs after myocardial infarction and often results in further decrease in ventricular function. In many cases, after a myocardial infarct heals, continued ischemia in the border region between the healed infarct and normal tissue and other factors lead to dilation and/or remodeling of the remaining heart tissue. This dilating or remodeling, while initially adaptive, often leads to further impairment of ventricular function. Dilation of the whole heart occurs in about 50% of patients who have such infarcts, and remodeling usually develops within a few months after a myocardial infarction although it can occur as early as 1-2 weeks after the infarct. Poor left ventricular function is the best single predictor of adverse outcome following myocardial infarction. Thus, preventing ventricular remodeling after myocardial infarction would be beneficial. One approach to try to prevent ventricular remodeling is to treat patients who have suffered a myocardial infarction with angiotensin converting enzyme (“ACE”) inhibitors (see, e.g., McDonald, K. M., Trans. Assoc. Am. Physicians 103: 229-235, 1990; Cohn, J. Clin. Cardiol. 18 (Suppl. IV) IV-4-IV-12, 1995). However, these agents are only somewhat effective at preventing deleterious ventricular remodeling and new therapies are needed.

Present treatments for CHF include pharmacological therapies, coronary revascularization procedures and heart transplantation. Pharmacological therapies for CHF have been directed toward increasing the force of contraction of the heart (by using inotropic agents such as digitalis and beta-adrenergic receptor agonists), reducing fluid accumulation in the lungs and elsewhere (by using diuretics), and reducing the work of the heart (by using agents that decrease systemic vascular resistance such as angiotensin converting enzyme inhibitors). Beta-adrenergic receptor antagonists have also been tested. While such pharmacological agents can improve symptoms, and potentially prolong life, the prognosis in most cases remains dismal.

Some patients with heart failure due to associated coronary artery disease can benefit, at least temporarily, by revascularization procedures such as coronary artery bypass surgery and angioplasty. Such procedures are of potential benefit when the heart muscle is not dead but may be dysfunctional because of inadequate blood flow. If normal coronary blood flow is restored, previously dysfunctional myocardium may contract more normally, and heart function may improve. However, if the patient has an inadequate microvascular bed (e.g., as may be found in more severe CHF patients), revascularization will rarely restore cardiac function to normal or near-normal levels, even though mild improvements are sometimes noted. In addition, the incidence of failed bypass grafts and restenosis following angioplasty poses further risks to patients treated by such methods. Heart transplantation can be a suitable option for CHF patients who have no other confounding diseases and are relatively young, but this is an option for only a small number of such patients, and only at great expense. In sum, it can be seen that CHF has a very poor prognosis and responds poorly to current therapies.

Further complicating the physiological conditions associated with CHF are various natural adaptations that tend to occur in patients with dysfunctional hearts. Although these natural responses can initially improve heart function, they often result in other problems that can exacerbate the disease, confound treatment, and have adverse effects on survival. There are three such adaptive responses commonly observed in CHF patients: (i) volume retention induced by changes in sodium reabsorption, which expands plasma volume and initially improves cardiac output; (ii) cardiac enlargement (from dilation and hypertrophy) which can increase stroke volume while maintaining a relatively normal wall tension; and (iii) increased norepinephrine release from adrenergic nerve terminals impinging on the heart which, by interacting with cardiac beta-adrenergic receptors, tends to increase heart rate and force of contraction, thereby increasing cardiac output. However, each of these three natural adaptations tends ultimately to fail for various reasons. In particular, fluid retention tends to result in edema and retained fluid in the lungs that impairs breathing. Heart enlargement can lead to deleterious left ventricular remodeling with subsequent severe dilation and increased wall tension, thus exacerbating CHF. Finally, long-term exposure of the heart to norepinephrine tends to make the heart unresponsive to adrenergic stimulation and is linked with poor prognosis.

Recently, investigations into treatments for cardiovascular disease have turned to therapeutics related to angiogenesis. Angiogenesis refers generally to the development and differentiation of blood vessels. A number of proteins, typically referred to as “angiogenic proteins,” are known to promote angiogenesis. Such angiogenic proteins include members of the fibroblast growth factor (FGF) family, the vascular endothelial growth factor (VEGF) family, the platelet-derived growth factor (PDGF) family, the insulin-like growth factor (IGF) family, and others (as described in more detail below and in the art). For example, the FGF and VEGF family members have been recognized as regulators of angiogenesis during growth and development. Their role in promoting angiogenesis in adult animals has recently been examined (as discussed below). The angiogenic activity of the FGF and VEGF families has bee examined. For example, it has been shown that acidic FGF (“aFGF”) protein, within a collagen-coated matrix, when placed in the peritoneal cavity of adult rats, resulted in a well vascularized and normally perfused structure (Thompson et al., Proc. Natl. Acad. Sci. USA, 86: 7928-7932, 1989). Injection of basic FGF (“bFGF”) protein into adult canine coronary arteries during coronary occlusion reportedly led to decreased myocardial dysfunction, smaller myocardial infarctions, and increased vascularity in the bed at risk (Yanagisawa-Miwa et al., Science, 257: 1401-1403, 1992). Similar results have been reported in animal models of myocardial ischemia using bFGF protein (Harada et al., J. Clin. Invest., 94: 623-630, 1994; Unger et al., Am. J. Physio., 266: H1588-H-1595, 1994). An increase in collateral blood flow was shown in dogs treated with VEGF protein (Banai et al. Circulation 89: 2183-2189, 1994).

However, difficulties associated with the potential use of such protein infusions to promote cardiac angiogenesis include: achieving proper localization for a sufficient period of time, and ensuring that the protein is and remains in the proper form and concentration needed for uptake and the promotion of an angiogenic effect within cells of the myocardium. A protein concentration which is high initially (e.g., following bolus infusion) but then drops rapidly (with clearance by the body) can be both toxic and ineffective. Another difficulty is the need for repeated infusion or injection of the protein.

Some publications postulated on the use of gene transfer for the treatment or prevention of disease, including certain heart diseases. See, for example, French, “Gene Transfer and Cardiovascular Disorders, “Herz 18: 222-229, 1993; Williams, “Prospects for Gene Therapy of Ischemic Heart Disease,” American Journal of Medical Sciences 306: 129-136, 1993; Schneider and French,” The Advent of Adenovirus: Gene Therapy for Cardiovascular Disease,” Circulation 88: 1937-1942, 1993; and Mazur et al., “Coronary Restenosis and Gene Therapy,” Molecular and Cellular Pharmacology, 21: 104-111, 1994.

Additionally, some groups have suggested in vivo gene transfer into the myocardium using plasmids, retrovirus, adenovirus and other vectors (see e.g., Barr et al., Supplement II, Circulation, 84 (4): Abstract 1673, 1991; Barr et al., Gene Ther., 1: 51-58, 1994; French et al., Circulation, 90 (5): 2402-2413, 1994; French et al., Circulation, 90 (5): 2414-2424, 1994; French et al., Circulation, 90: 1517 Abstract No. 2785, 1994; Leiden, et al., WO94/11506 (26 May 1994); Guzman et al., Circ. Res., 73 (6): 1202-1207, 1993; Kass-Eisler et al., Proc. Natl. Acad. Sci. USA, 90: 11498-11502, 1993; Miihlhauser et al., Hum. Gene Ther., 6: 1457-1465, 1995; Miihlhauser et al. Circ. Res., 77 (6): 1077-1086, 1995; and Rowland et al., Am. Thorac. Surg., 60 (3): 721-728, 1995.

In general, however, these reports provided little more than suggestions or wishes for potential therapies. Of those providing animal data, most did not employ disease models suitably related to actual in vivo conditions. Moreover, the attempted in vivo methods generally suffered from one or more of the following deficiencies: inadequate transduction efficiency and transgene expression; marked immune response to the vectors used, including inflammation and tissue necrosis; and importantly, a relative inability to target transduction and transgene expression to the organ of interest (e.g., gene transfer targeted to the heart resulted in the transgene also being delivered to non-cardiac sites such as liver, kidneys, lungs, brain and testes of the test animals). By way of example, the insertion of a transgene into a rapidly dividing cell population will result in substantially reduced duration of transgene expression. Examples of such cells include endothelial cells, which make up the inner layer of all blood vessels, and fibroblasts, which are dispersed throughout the heart. Targeting the transgene so that only the desired cells will receive and express the transgene, and so that the transgene will not be systemically distributed, are also critically important considerations. If this is not accomplished, systemic expression of the transgene and problems attendant thereto will result. For example, inflammatory infiltrates have been documented after adenovirus-mediated gene transfer in liver (Yang et al. Proc. Natl. Acad. Sci. U.S.A, 91: 4407, 1994). Additionally, inflammatory infiltrates were documented in the heart after direct intramyocardial injection through a needle inserted into the myocardial wall (French et al., Circulation, 90 (5): 2414-2424, 1994).

At present there is a need, provided for in the present invention, for improved and more efficient methods of gene transfer to targeted tissues in order achieve the amount of gene expression necessary for a therapeutic effect and in order to reduce toxicity and inflammatory responses.

### SUMMARY OF THE INVENTION

The present invention relates to methods of delivering a vector comprising a nucleic acid to the myocardium of a patient, comprising the steps of: (a) inflating a balloon catheter within a coronary artery supplying blood to the myocardium to cause a first occlusion of myocardial blood flow, (b) allowing a first reperfusion of the myocardium by deflating the balloon catheter to release the first occlusion, (c) reinflating the balloon catheter to cause a second occlusion of myocardial blood flow, and (d) infusing a solution comprising the vector into the coronary artery downstream of the site of the second occlusion, and (e) allowing a second reperfusion of the myocardium by deflating the balloon catheter to release the second occlusion. The method may be repeated in a second coronary artery of the patient.

In a preferred embodiment of the invention, said step of infusing a solution comprising the vector into the coronary artery is performed coincident with the second occlusion by infusing the solution through an infusion port of the balloon catheter. In another embodiment of the invention, said step of infusing a solution comprising the vector into the coronary artery is performed coincident with the second reperfusion of the myocardium by infusing the solution into the myocardial blood flow after release of the second occlusion.

The first occlusion preferably is at least one minute in duration but extended to several minutes provided that the patient does not exhibit signs of acute ischemia. In a more preferred embodiment of the invention, the first occlusion is approximately two to three minutes. The occlusion is preferably released as soon as possible following observation of an indicator of acute ischemia. Indicators of acute ischemia are known in the art and include chest pain, a drop in blood pressure, ST segment depression, and tachycardia.

The first reperfusion is preferably between one and ten minutes in duration. In a more preferred embodiment of the invention, the first reperfusion is approximately five minutes.

In a preferred embodiment of the invention, the second occlusion is between one and five minutes in duration. In a more preferred embodiment of the invention, the second occlusion is approximately three minutes.

In a preferred embodiment of the invention, the method includes the additional step of infusing a vasoactive agent. The vasoactive agent preferably is infused during at least a portion of the first reperfusion. In a still more preferred embodiment, the vasoactive agent is infused during a portion of the first reperfusion and during a portion of the second occlusion by infusing the solution through an infusion port of the balloon catheter.

In an embodiment of the invention, the vasoactive agent is selected from the group consisting of histamine, a histamine agonist, sodium nitroprusside (SNP), an SNP agonist or a vascular endothelial growth factor. In a more preferred embodiment, the vasoactive agent is nitroglycerin.

In a preferred embodiment of the invention, the first occlusion is approximately two to three minutes and the first reperfusion is approximately five minutes. In a preferred embodiment, a vasoactive agent is infused beginning approximately two minutes after initiation of the second occlusion and is continued throughout the second occlusion.

In a preferred embodiment of the invention, the solution comprising the vector is infused during the second occlusion. Preferably, the solution comprising the vector is slowly infused over a period of at least thirty seconds during the second occlusion. More preferably, the solution comprising the vector is slowly infused over approximately ninety seconds during the second occlusion. The rate of infusion is preferably less than 10 ml per minute, more preferably less than 5 ml per minute, still more preferably less than 2 ml per minute. In an exemplary embodiment, the solution comprising the vector is infused at a rate of 2 ml per minute.

The vector may be viral or a non-viral vector that facilitates the delivery of the nucleic acid to the myocardium. In a preferred viral vector is a replication-deficient adenovirus.

The vector concentration can be optimized to achieve a desired degree of expression. In the case of adenovirus, the solution comprising the vector contains between about 10E7 to about 10E13 adenovirus vector particles. In a preferred embodiment, the solution comprising the vector contains between about 10E9 to about 10E11 adenovirus vector particles.

The nucleic acid may encode a protein that is desired to be expressed in the myocardium. In addition to many other types of proteins, the protein may be a zinc finger DNA binding protein which can be used to modulate the expression of endogenous genes within the cell of the heart. In a preferred embodiment, the protein is selected from the group consisting of a factor which induces angiogenesis, a factor which promotes myocardial contractility, a factor which promotes cardiac cell survival, and a factor which recruits cells to or within or to the heart, such as cardiac stem cells. In an illustrative embodiment, the protein is an angiogenic or other growth factor. In a more preferred embodiment the protein is selected from the group consisting of a fibroblast growth factor, a vascular endothelial growth factor, a platelet-derived growth factor, a hypoxia-inducible factor, an angiogenic polypeptide regulator, and an insulin-like growth factor. In a more preferred embodiment, the protein is human fibroblast growth factor (hFGF). In an illustrative embodiment the protein is hFGF type IV.

The nucleic acid may contain a gene controlled by its own homologous promoter or alternately it may be operably linked to a heterologous promoter. The heterologous promoter may be a constitutive promoter or it may be an inducible promoter depending on the desired levels and timing of expression. In an exemplary embodiment, the heterologous promoter is a cytomegalovirus (CMV) promoter.

In a preferred embodiment of the invention the nucleic acid encodes a protein expressed in the heart. In an exemplary embodiment, the expression of the protein in the heart results in angiogenesis, increased myocardial contractility, promotion of cardiac cell survival, or recruitment of cells within or to the heart. In a more preferred embodiment the expression of the protein in the heart also results in an increase of myocardial perfusion.

In another embodiment of the invention, the nucleic acid also encodes a second protein. In illustrative embodiment, the second protein may be selected from the group consisting of a factor which induces angiogenesis, a factor which promotes myocardial contractility, a factor which promotes cardiac cell survival, and a factor which recruits cells within or to the heart, including cardiac stem cells.

The invention can be used to treat a patient suffering from any of a variety of conditions affecting the heart such as atherosclerosis, the occurrence of one or more In another embodiment, the invention is used treat a patient having one or more myocardial perfusion deficits when the heart is imaged under stress.

In a preferred embodiment, the invention is used to treat a patient having myocardial ischemia.

In another embodiment, the invention is used to treat a patient that has experienced angina pectoris.

In a preferred embodiment, the invention is used to treat a patient with congestive heart failure.

The invention also relates to the use of a vector comprising a nucleic acid in the preparation of a medicament for the treatment of heart disease, wherein the medicament is for infusion into the myocardium through introduction into a coronary artery following induced occlusion.

The invention also relates to the use of a vector comprising a nucleic acid in the preparation of a medicament for the treatment of heart disease, wherein the medicament is for infusion into the myocardium through introduction into a coronary artery following induced occlusion, wherein the infusion into the myocardium is in accordance with the methods of the present invention.

The invention also relates to a combination comprising a medicament for the treatment of heart disease, wherein the medicament is for infusion into the myocardium through introduction into a coronary artery following induced occlusion, and a device for the occlusion of a coronary artery.

The invention also relates to a combination comprising a medicament for the treatment of heart disease, wherein the medicament is for infusion into the myocardium through introduction into a coronary artery following induced occlusion, and a device for the occlusion of a coronary artery. In a preferred embodiment, the device for the occlusion of a coronary artery is a balloon catheter. In a more preferred embodiment, the balloon catheter comprises an infusion port through which a solution may be infused while the balloon is inflated. In a more preferred embodiment, the induced occlusion comprises a first and second occlusion of the coronary artery separated by a first reperfusion. In a more preferred embodiment, the induced occlusion comprises the steps of: (a) inflating a balloon catheter within a coronary artery supplying blood to the myocardium to cause a first occlusion of myocardial blood flow, (b) allowing a first reperfusion of the myocardium by deflating the balloon catheter to release the first occlusion, (c) reinflating the balloon catheter to cause a second occlusion of myocardial blood flow, and (d) infusing a solution comprising the vector into the coronary artery downstream of the site of the second occlusion, and (e) allowing a second reperfusion of the myocardium by deflating the balloon catheter to release the second occlusion.

The invention also relate to a combination comprising a medicament for the treatment of heart disease, wherein the medicament is for infusion into the myocardium through introduction into a coronary artery following induced occlusion, and a device for the occlusion of a coronary artery, wherein the induced occlusion is performed in accordance with the methods of the present invention.

### DETAILED DESCRIPTION OF PREFERRED EMBODIMENTS

In the context of the present application and invention, the following definitions apply.

“Heart disease” refers to acute and/or chronic cardiac dysfunctions. Heart disease is often associated with a decrease in cardiac contractile function and may be associated with an observable decrease in blood flow to the myocardium (e.g., as a result of coronary artery disease). Manifestations of heart disease include myocardial ischemia, which may result in angina, heart attack and/or congestive heart failure.

“Myocardial ischemia” is a condition in which the heart muscle does not receive adequate levels of oxygen and nutrients, which is typically due to inadequate blood supply to the myocardium (e.g., as a result of coronary artery disease).

“Heart failure” is clinically defined as a condition in which the heart does not provide adequate blood flow to the body to meet metabolic demands. Symptoms include breathlessness, fatigue, weakness, leg swelling, and exercise intolerance. On physical examination, patients with heart failure tend to have elevations in heart and respiratory rates, rales (an indication of fluid in the lungs), edema, jugular venous distension, and, in many cases, enlarged hearts. Patients with severe heart failure suffer a high mortality; typically 50% of the patients die within two years of developing the condition. In some cases, heart failure is associated with severe coronary artery disease (“CAD”), typically resulting in myocardial infarction and either progressive chronic heart failure or an acute low output state, as described herein and in the art. In other cases, heart failure is associated with dilated cardiomyopathy without associated severe coronary artery disease.

As used herein, the terms “having therapeutic effect” and “successful treatment” carry essentially the same meaning. In particular, a patient suffering from heart disease is successfully “treated” for the condition if the patient shows observable and/or measurable reduction in or absence of one or more of the symptoms of heart disease after receiving an angiogenic factor transgene according to the methods of the present invention. Reduction of these signs or symptoms may also be felt by the patient. Thus, indicators of successful treatment of heart disease conditions include the patient showing or feeling a reduction in any one of the symptoms of angina pectoris, fatigue, weakness, breathlessness, leg swelling, rales, heart or respiratory rates, edema or jugular venous distension. The patient may also show greater exercise tolerance, have a smaller heart with improved ventricular and cardiac function, and in general, require fewer hospital visits related to the heart condition. The improvement in cardiovascular function may be adequate to meet the metabolic needs of the patient and the patient may not exhibit symptoms under mild exertion or at rest. Many of these signs and symptoms are readily observable by eye and/or measurable by routine procedures familiar to a physician. Indicators of improved cardiovascular function include increased blood flow and/or contractile function in the treated tissues. As described below, blood flow in a patient can be measured by thallium imaging (as described by Braunwald in Heart Disease, 4th ed., pp. 276-311 (Saunders, Philadelphia, 1992)) or by echocardiography (described in Examples 1 and 5 and in Sahn, Di., et al., Circulation. 58: 1072-1083, 1978). Blood flow before and after angiogenic gene transfer can be compared using these methods. Improved heart function is associated with decreased signs and symptoms, as noted above. In addition to echocardiography, one can measure ejection fraction (LV) by nuclear (non-invasive) techniques as is known in the art.

An “angiogenic protein or peptide” refers to any protein or peptide capable of promoting angiogenesis or angiogenic activity, i. e. blood vessel development.

A “polynucleotide” refers to a polymeric form of nucleotides of any length, either ribonucleotides or deoxyribonucleotides, or analogs thereof. This term refers to the primary structure of the molecule, and thus includes double- and single-stranded DNA, as well as double- and single-stranded RNA including siRNA and microRNA. It also includes modified polynucleotides such as methylated and/or capped polynucleotides.

“Recombinant,” as applied to a polynucleotide, means that the polynucleotide is the product of various combinations of cloning restriction and/or ligation steps, and other procedures that result in a construct that is distinct from a polynucleotide found in nature.

A “gene” or “transgene” refers to a polynucleotide or portion of a polynucleotide comprising a sequence that encodes a protein. For most situations, it is desirable for the gene to also comprise a promoter operably linked to the coding sequence in order to effectively promote transcription. Enhancers, repressors and other regulatory sequences may also be included in order to modulate activity of the gene, as is well known in the art. (See, e.g., the references cited below).

The terms “polypeptide,” “peptide,” and “protein” are used interchangeably to refer to polymers of amino acids of any length. These terms also include proteins that are post-translationally modified through reactions that include glycosylation, acetylation and phosphorylation.

A “heterologous” component refers to a component that is introduced into or produced within a different entity from that in which it is naturally located. For example, a polynucleotide derived from one organism and introduced by genetic engineering techniques into a different organism is a heterologous polynucleotide which, if expressed, can encode a heterologous polypeptide. Similarly, a promoter or enhancer that is removed from its native coding sequence and operably linked to a different coding sequence is a heterologous promoter or enhancer.

A “promoter,” as used herein, refers to a polynucleotide sequence that controls transcription of a gene or coding sequence to which it is operably linked. A large number of promoters, including constitutive, inducible and repressible promoters, from a variety of different sources, are well known in the art (and identified in databases such as GenBank) and are available as or within cloned polynucleotide sequences (from, e.g., depositories such as the ATCC as well as other commercial or individual sources).

An “enhancer,” as used herein, refers to a polynucleotide sequence that enhances transcription of a gene or coding sequence to which it is operably linked. A large number of enhancers, from a variety of different sources are well known in the art (and identified in databases such as GenBank) and available as or within cloned polynucleotide sequences (from, e.g., depositories such as the ATCC as well as other commercial or individual sources). A number of polynucleotides comprising promoter sequences (such as the commonly-used CMV promoter) also comprise enhancer sequences.

“Operably linked” refers to a juxtaposition of two or more components, wherein the components so described are in a relationship permitting them to function in their intended manner. A promoter is operably linked to a gene or coding sequence if the promoter controls transcription of the gene or coding sequence. Although an operably linked promoter is generally located upstream of the coding sequence, it is not necessarily contiguous with it. An enhancer is operably linked to a coding sequence if the enhancer increases transcription of the coding sequence. Operably linked enhancers can be located upstream, within or downstream of coding sequences. A polyadenylation sequence is operably linked to a coding sequence if it is located at the downstream end of the coding sequence such that transcription proceeds through the coding sequence into the polyadenylation sequence.

A “replicon” refers to a polynucleotide comprising an origin of replication which allows for replication of the polynucleotide in an appropriate host cell. Examples include chromosomes of a target cell into which a heterologous nucleic acid might be integrated (e.g., nuclear and mitochondrial chromosomes), as well as extrachromosomal replicons (such as replicating plasmids and episomes).

“Gene delivery”, “gene transfer,” and the like as used herein, are terms referring to the introduction of an exogenous polynucleotide (sometimes referred to as a “transgene”) into a host cell, irrespective of the method used for the introduction. Such methods include a variety of well-known techniques such as vector-mediated gene transfer (by, e.g., viral infection/transfection, or various other protein-based or lipid-based gene delivery complexes) as well as techniques facilitating the delivery of “naked” polynucleotides (such as electroporation, “gene gun” delivery and various other techniques used for the introduction of polynucleotides). The introduced polynucleotide may be stable or transiently maintained in the host cell. Stable maintenance typically requires that the introduced polynucleotide either contains an origin of replication compatible with the host cell or integrates into a replicon of the host cell such as an extrachromosomal replicon (e.g., a plasmid) or a nuclear or mitochondrial chromosome. A number of vectors are known to be capable of mediating transfer of genes to mammalian cells, as is known in the art and described herein.

“In vivo” gene delivery, gene transfer, gene therapy and the like as used herein, are terms referring to the introduction of a vector comprising an exogenous polynucleotide directly into the body of an organism, such as a human or non-human mammal, whereby the exogenous polynucleotide is introduced into a cell of such organism in vivo.

A “vector” (sometimes referred to as a gene delivery or gene transfer “vehicle”) refers to a macromolecule or complex of molecules comprising a polynucleotide to be delivered to a host cell, either in vitro or in vivo. The polynucleotide to be delivered may comprise a coding sequence of interest in gene therapy.

“Vasculature” or “vascular” are terms referring to the system of vessels carrying blood (as well as lymph fluids) throughout the mammalian body.

“Blood vessel” refers to any of the vessels of the mammalian vascular system, including arteries, arterioles, capillaries, venues, veins, sinuses, and vasa vasorum. In preferred aspects of the present invention for treating heart disease, vectors comprising nucleic acids are introduced directly into vascular conduits supplying blood to the myocardium. Such vascular conduits include the coronary arteries as well as vessels such as saphenous veins or internal mammary artery grafts.

“Artery” refers to a blood vessel through which blood passes away from the heart. Coronary arteries supply the tissues of the heart itself, while other arteries supply the remaining organs of the body. The general structure of an artery consists of a lumen surrounded by a multi-layered arterial wall.

An “individual” or a “patient” refers to a mammal, preferably a large mammal, most preferably a human.

“Treatment” or “therapy” as used herein refers to administering, to an individual patient, agents that are capable of eliciting a prophylactic, curative or other beneficial effect on the individual.

“Gene therapy” as used herein refers to administering, to an individual patient, vectors comprising a therapeutic gene or genes.

A “therapeutic polynucleotide” or “therapeutic gene” refers to a nucleotide sequence that is capable, when transferred to an individual, of eliciting a prophylactic, curative or other beneficial effect in the individual.

“Vasoactive agent,” as used herein, refers to a natural or synthetic substance that induces increased vascular permeability and/or enhances transfer of macromolecules such as gene delivery vectors from blood vessels, e.g. across capillary endothelia. By augmenting vascular permeability to macromolecules or otherwise facilitating the transfer of macromolecules into the capillary bed perfused by an artery, vasoactive agents can enhance delivery of these vectors to the targeted sites and thus effectively enhance overall expression of the transgene in the target tissue.

“Intermittent occlusion” as used herein, refers to the occlusion of an artery such that blood is substantially unable to flow past the occlusion, except through collateral blood vessels, if such blood vessels exist, and then a release of the occlusion allowing reperfusion into the artery; as illustrated herein.

### REFERENCES

The practice of the present invention will employ, unless otherwise indicated, conventional techniques of molecular biology and the like, which are within the skill of the art. Such techniques are explained in the literature. See e.g., Molecular Cloning: A Laboratory Manual (Sambrook et al., Cold Spring Harbor Laboratory, Cold Spring Harbor, N.Y., 1989); Current Protocols in Molecular Biology (Ausubel et al., 1987 and updated 2002); Essential Molecular Biology (Brown, IRL Press 2000); Gene Expression Technology (Goeddel, Academic Press 1991); Methods for Cloning and Analysis of Eukaryotic Genes (Bothwell et al., Bartlett Publ. 1990); Gene Transfer and Expression (Kriegler, Stockton Press 1990); Recombinant DNA Methodology (Wu et al., Academic Press 1989); PCR: A Practical Approach (McPherson, IRL Press at Oxford University Press 1995); Cell Culture for Biochemists (Adams, Elsevier Science Publishers 1990); Gene Transfer Vectors for Mammalian Cells (Miller et al., 1987); Mammalian Cell Biotechnology (Butler, 1991); Animal Cell Culture (Pollard et al., Humana Press 1990); Culture of Animal Cells, 4th Ed. (Freshney et al., Alan R. Liss, 2000); Flow Cytometry and Sorting (Melamed et al., Wiley-Liss 1990); the series Methods in Enzymology (Academic Press, Inc.); Techniques in Immunocytochemistry (Bullock et al., Academic Press 1982, 1983, 1985, 1989); Handbook of Experimental Immunology (Weir, Blackwell, 5th ed., 1996); Cellular and Molecular Immunology (Abbas et al., W.B. Saunders Co. 5th ed., 2003); Current Protocols in Immunology (Coligan et al., 1996); the series Annual Review of Immunology; the series Advances in Immunology; Oligonucleotide Synthesis (Gait, 1984); and Animal Cell Culture (Freshney, IRL Press 1992, 2nd ed.).

Additional references describing delivery and logistics of surgery which may be used in the methods of the present invention include the following: The Textbook of Interventional Cardiology, 4th ed. (Topol, W.B. Saunders Co. 2002); Vascular Surgery, 5th ed. (Rutherford, W.B. Saunders Co. 2000); The Cecil Textbook of Medicine, 22nd Ed. (Elsevier, Inc. 2004); and Sabiston Textbook of Surgery, 17th Ed. (Elsevier, Inc. 2004). Additional references describing cell types found in the blood vessels, and those of the vasculature which may be useful in the methods of the present invention include, for example, A Textbook of Histology (Bloom & Fawcett, W.B. Saunders Co. 1975).

Various publications have postulated on the uses of gene transfer for the prevention of disease, including heart disease, and are referenced herein. Additionally, see, for example, Methods in Virology, Vol. 7: Gene Transfer and Expression Protocols, Murray (ed.), Weiss, Clifton, N.J., 1991; Mazur et al. (1994) Mol. Cell. Biol. 21:104-111; French (1993) Herz. 18:222-229; Williams (1993) Am. J. Med. Sci. 306:129-136; and Schneider et al. (1993) Circulation 88:1937-1942. Similarly, various publications describe various vectors that may be useful in gene therapy methods. See, e.g., Hammond et al., WO 96/26742; Hammond, WO 02/089856, hereby incorporated by reference in its entirety; Flotte et al., WO 95/13365; Trempe et al., WO 95/13392; Gnatenko (1997) J. of Invest. Med. 45:87-97; and other references cited herein

The references cited in the above section are hereby incorporated by reference herein to the extent that these references teach techniques that are employed in the practice of the present invention.

All references cited within this application, including patents, patent applications and other publications, are hereby incorporated by reference.

### DETAILED DESCRIPTION OF VARIOUS PREFERRED EMBODIMENTS

Various preferred aspects of the present invention are summarized below.

The present invention relates to methods and compositions for treating cardiovascular diseases including myocardial ischemia, and heart failure.

In the present method, for treating heart disease, a vector construct containing a gene encoding a protein or peptide to be targeted to the heart of a patient whereby the exogenous angiogenic protein is expressed in the myocardium, thus ameliorating cardiac dysfunction; for example by improving blood flow and/or improving cardiac contractile function. Improved heart function ultimately leads to the reduction or disappearance of one or more symptoms of heart disease or heart failure and prolonged life beyond the expected mortality.

In an exemplary embodiment, the present invention provides a method for treating heart disease in a patient having myocardial ischemia, comprising delivering a transgene-inserted vector to the myocardium of the patient by intracoronary injection, preferably by injecting the vector directly into one or both coronary arteries (or grafts), whereby the transgene is expressed and blood flow and/or contractile function are improved. By way of illustration, using a vector comprising a transgene coding for an angiogenic protein or peptide, such as, for example, FGF-5, FGF-4, aFGF, bFGF and/or a VEGF, which vector is delivered to the heart where the protein or peptide is produced to a therapeutically significant degree in the myocardium continuously for sustained periods, angiogenesis can be promoted in the affected region of the myocardium. Other transgenes, such as those encoding beta-adrenergic signaling proteins or other cardiac- or muscle-enhancing proteins, can also be used, as described below, in conjunction with the use of an angiogenic transgene.

In another preferred aspect, the present invention can also be used to treat a patient suffering from congestive heart failure, by delivering a transgene-inserted vector to the heart of said patient, the vector comprising a transgene encoding a, whereby the transgene is expressed in the myocardium resulting in increased blood flow and function in the heart. Among such patients suffering from congestive heart failure are those exhibiting dilated cardiomyopathy and those who have exhibited severe myocardial infarctions, typically associated with severe or occlusive coronary artery disease. The vector is preferably introduced into a blood vessel supplying blood to the myocardium of the heart, so as to deliver the vector to the myocardium. Preferably the vector is introduced into the lumen of a coronary artery, a saphenous vein graft, or an internal mammary artery graft; most preferably, the vector is introduced into the lumen of both a left and right coronary artery according to the methods described herein.

The techniques of the present invention are also useful to prevent or alleviate deleterious ventricular remodeling in a patient who has suffered (or may suffer) a myocardial infarction. Again, a vector comprising a transgene encoding an angiogenic protein or peptide, preferably operably linked to a promoter for expression of the gene, is delivered to the heart of the patient, where the transgene is expressed and the deleterious ventricular remodeling alleviated.

In one aspect, the vectors and methods of the present invention can be employed to treat dilated cardiomyopathy (DCM), a type of heart failure that is typically diagnosed by the finding of a dilated, hypocontractile left and/or right ventricle. As discussed above, DCM can occur in the absence of other characteristic forms of cardiac disease such as coronary occlusion or a history of myocardial infarction. DCM is associated with poor ventricular function and symptoms of heart failure. In these patients, chamber dilation and wall thinning generally results in a high left ventricular wall tension. Many patients exhibit symptoms even under mild exertion or at rest, and are thus characterized as exhibiting severe, i. e. “Type-m” or “Type-IV”, heart failure, respectively (see, e.g., NYHA classification of heart failure). As noted above, many patients with coronary artery disease may progress to exhibiting dilated cardiomyopathy, often as a result of one or more heart attacks (myocardial infarctions).

Methods of assessing improvement in heart function and reduction of symptoms are essentially analogous to those described above for DCM. Prevention or alleviation of deleterious ventricular remodeling as a result of improved collateral blood flow and ventricular function and/or other mechanisms is expected to be achieved within weeks after in vivo angiogenic gene transfer in the patient using methods as described herein.

In treating angina, as may be associated with CAD, gene transfer of an angiogenic protein encoded by a transgene can be conducted at any time, but preferably is performed relatively soon after the onset of severe angina. In treating most congestive heart failure, gene transfer of an angiogenic protein encoding transgene can be conducted, for example, when development of heart failure is likely or heart failure has been diagnosed. For treating ventricular remodeling, gene transfer can be performed any time after the patient has suffered an infarct, preferably within 30 days and even more preferably within 7-20 days after an infarct.

Transgenes that may be used with the present invention.

The present invention can be used to achieve the delivery and expression of any variety of genes to the heart of a patient. By way of illustration, genes that can be used to alter the physiology of the heart in a desired manner, which may be referred to a “cardioactive genes” include genes used to encode proteins which induce angiogenesis, which promote myocardial contractility, which promote cardiac cell survival, which recruit cells within or to the heart, such as cardiac stem cells, or which otherwise induce or modulate the physiology of the heart or portions thereof. Numerous such cardioactive genes are well known in the art and new cardioactive genes are regularly being identified. By way of illustration of the principles and techniques of the present invention, one or more transgenes encoding an angiogenic protein or peptide factor that can enhance blood flow and/or contractile function can be used. Any protein or peptide that exhibits angiogenic activity, measurable by the methods described herein and in the art, can be potentially employed in connection with the present invention. A number of such angiogenic proteins are known in the art and new forms are routinely identified. Suitable angiogenic proteins or peptides are exemplified by members of the family of fibroblast growth factors (FGF), vascular endothelial growth factors (VEGF), platelet-derived growth factors (PDGF), insulin-like growth factors (IGF), and others. Members of the FGF family include, but are not limited to, aFGF (FGF-1), bFGF (FGF-2), FGF-4 (also known as “hst/KS3”), FGF-5, and FGF-6. VEGF has been shown to be expressed by cardiac myocytes in response to ischemia in vitro and in vivo; it is a regulator of angiogenesis under physiological conditions as well as during the adaptive response to pathological states (Banai et al. Circulation 89: 2183-2189, 1994). The VEGF family, includes, but is not limited to, members of the VEGF-A sub-family (e.g. VEGF-121, VEGF-145, VEGF-165, VEGF-189 and VEGF-206), as well as members of the VEGF-B sub-family (e.g. VEGF-167 and VEGF-186) and the VEGF-C sub-family. PDGF includes, e.g., PDGF A and PDGF B, and IGF includes, for example, IGF-1. Other angiogenic proteins or peptides are known in the art and new ones are regularly identified. The nucleotide sequences of genes encoding these and other proteins, and the corresponding amino acid sequences are likewise known in the art (see, e.g., the GENBANK sequence database).

Angiogenic proteins and peptides include peptide precursors that are post-translationally processed into active peptides and “derivatives” and “functional equivalents” of angiogenic proteins or peptides. Derivatives of an angiogenic protein or peptide are peptides having similar amino acid sequence and retaining, to some extent, one or more activities of the related angiogenic protein or peptide. As is well known to those of skill in the art, useful derivatives generally have substantial sequence similarity (at the amino acid level) in regions or domains of the protein associated with the angiogenic activity. Similarly, those of skill in the art will readily appreciate that by “functional equivalent” is meant a protein or peptide that has an activity that can substitute for one or more activities of a particular angiogenic protein or peptide. Preferred functional equivalents retain all of the activities of a particular angiogenic protein or peptide; however, the functional equivalent may have an activity that, when measured quantitatively, is stronger or weaker than the wild-type peptide or protein.

For details on the FGF family, see, e.g., Burgess, Ann. N. Y. Acad. Sci. 638: 89-97, 1991; Burgess et al. Annu. Rev. Biochem. 58: 575-606, 1989; Muhlhauser et al., Hum. Gene Ther. 6: 1457-1465, 1995; Zhan et al., Mol. Cell. Biol., 8: 3487, 1988; Seddon et al., Ann. N. Y. Acad. Sci. 638: 98-108, 1991. For human hst/KS3 (i. e. FGF-4), see Taira et al. Proc. Natl. Acad. Sci. USA 84: 2980-2984, 1987. For human VEGF-A protein, see e.g., Tischer et al. J. Biol. Chem. 206: 11947-11954, 1991, and references therein; Muhlhauser et al., Circ. Res. 77: 1077-1086, 1995; and Neufeld et al., WO 98/10071 (12 Mar. 1998). Other variants of known angiogenic proteins have likewise been described; for example variants of VEGF proteins and VEGF related proteins, see e.g., Baird et al., WO 99/40197, (12 Aug. 1999); and Bohlen et al., WO 98/49300, (5 Nov. 1998). Combinations of angiogenic proteins and gene delivery vectors encoding such combinations are described in Gao et al. U.S. Ser. No. 09/607,766, filed 30 Jun. 2000, entitled “Dual Recombinant Gene Therapy Compositions and Methods of Use”, hereby incorporated by reference in its entirety. As is also appreciated by those of skill in the art, angiogenic proteins can promote angiogenesis by enhancing the expression, stability or functionality of other angiogenic proteins. Examples of such angiogenic proteins or peptides include, e.g., regulatory factors that are induced in response to hypoxia (e.g. the hypoxia-inducible factors such as Hif-1, Hif-2 and the like; see, e.g., Wang et al., Proc. Natl. Acad. Sci. USA 90 (9): 4304-8, 1993; Forsythe et al., Mol. Cell. Biol. 16 (9): 4604-13, 1996; Semenza et al., Kidney Int., 51 (2): 553-5, 1997; and O'Rourke et al., Oncol. Res., 9 (6-7): 327-32, 1997; as well as other regulatory factors, such as, for example, those that are induced by physiological conditions associated with cardiovascular disease, such as inflammation (e.g., inducible nitric oxide synthase (iNOS), as well as the constitutive counterpart, cNOS; see e.g., Yoshizumi et al., Circ. Res., 73 (1): 205-9, 1993; Chartrain et al., J. Biol. Chem., 269 (9): 6765-72, 1994; Papapetropoulos et al., Am. J. Pathol., 150 (5): 1835-44, 1997; and Palmer, et al., Am. J. Physio., 274 (2 Pt 1): L212-9, 1998). Additional examples of such angiogenic proteins include certain insulin-like growth factors (e.g., IGF-1) and angiopoietins (Angs), which have been reported to promote and/or stimulate expression and/or activity of other angiogenic proteins such as VEGF (see e.g. Goad, et al, Endocrinology, 137 (6): 2262-68 (1996); Warren, et al., J. Bio. Chem., 271 (46): 29483-88 (1996); Punglia, et al, Diabetes, 46 (10): 1619-26 (1997); and Asahara, et al., Circ. Res., 83 (3): 233-40 (1998) and Bermont et al. Int. J. Cancer 85: 117-123, 2000). Similarly, hepatocyte growth factor (also referred to as Scatter factor), which has been reported to induce blood vessel formation in vivo (see, e.g., Grant et al. Proc. Natl. Acad. Sci. USA 90: 1937-1941, 1993) has also been reported to increase expression of VEGF (see, e.g., Wojta et al., Lab Invest. 79: 427-438, 1999). Additional examples of angiogenic polypeptides include natural and synthetic regulatory peptides (angiogenic polypeptide regulators) that act as promoters of endogenous angiogenic genes. Native angiogenic polypeptide regulators can be derived from inducers of endogenous angiogenic genes. Hif, as described above, is one illustrative example of such an angiogenic gene which has been reported to promote angiogenesis by inducing expression of other angiogenic genes. Synthetic angiogenic polypeptide regulators can be designed, for example, by preparing multi-finger zinc-binding proteins that specifically bind to sequences upstream of the coding regions of endogenous angiogenic genes and which can be used to induce the expression of such endogenous genes. Studies of numerous genes has led to the development of “rules” for the design of such zinc-finger DNA binding proteins (see, e.g., Rhodes and Klug, Scientific American, February 1993, pp 56-65; Choo and Klug, Proc. Natl. Acad. Sci. USA, 91 (23): 11163-7, 1994; Rebar and Pabo, Science, 263 (5147): 671-3, 1994; Choo et al., J. Mol. Biol., 273 (3): 525-32, 1997; Pomerantz et al., Science 267: 93-96, 1995; and Liu et al., Proc. Natl. Acad. Sci. USA, 94: 5525-5530, 1997. As will be appreciated by those of skill in the art, numerous additional genes encoding proteins or peptides having the capacity to directly or indirectly promote angiogenesis are regularly identified and new genes will be identified based on similarities to known angiogenic protein or peptide encoding genes or to the discovered capability of such genes to encode proteins or peptides that promote angiogenesis. Sequence information for such genes and encoded polypeptides is readily obtainable from sequence databases such as GenBank or EMBL. Polynucleotides encoding these proteins can also be obtained from gene libraries, e.g., by using PCR or hybridization techniques routine in the art.

Preferably, the protein-encoding transgene is operably linked to a promoter that directs transcription and expression of the gene in a mammalian cell, such as a cell in the heart or in the skeletal muscle. One presently preferred promoter is a CMV promoter. In other preferred embodiments, as discussed further below, the promoter is a tissue-specific promoter, such as a cardiac-specific promoter (e.g., a cardiomyocyte-specific promoter). Preferably, the gene encoding the protein is also operably linked to a polyadenylation signal.

Success of the gene transfer approach requires both synthesis of the gene product and secretion from the transfected cell. Thus, preferred angiogenic proteins or peptides include those which are naturally secreted or have been modified to permit secretion, such as by operably linking to a signal peptide. From this point of view, a gene encoding a secreted angiogenic protein, such as, FGF-4, FGF-5, or FGF-6 is preferred since these proteins contain functional secretory signal sequences and are readily secreted from cells. Many if not most human VEGF proteins (including but not limited to VEGF-121 and VEGF-165) also are readily secreted and diffusible after secretion. Thus, when expressed, these angiogenic proteins can readily access the cardiac interstitium and induce angiogenesis. Blood vessels that develop in angiogenesis include capillaries which are the smallest caliber blood vessels having a diameter of about 8 microns, and larger caliber blood vessels that have a diameter of at least about 10 microns. Angiogenic activity can be determined by measuring blood flow, increase in function of the treated tissue or the presence of blood vessels, using procedures known in the art or described herein. For example, capillary number or density can be quantitated in an animal visually or by microscopic analysis of the tissue site (see Example 5).

With other angiogenic proteins such as aFGF (FGF-1) and bFGF (FGF-2) that lack a native secretory signal sequence, fusion proteins having secretory signal sequences can be recombinantly produced using standard recombinant DNA methodology familiar to one of skill in the art. It is believed that both aFGF and bFGF are naturally secreted to some degree; however, inclusion of an additional secretion signal sequence can be used to enhance secretion of the protein. The secretory signal sequence would typically be positioned at the N-terminus of the desired protein but can be placed at any position suitable to allow secretion of the angiogenic factor. For example, a polynucleotide containing a suitable signal sequence can be fused to the first codon of the selected angiogenic protein gene. Suitable secretory signal sequences include signal sequences of the FGF-4, FGF-5, FGF-6 genes or a signal sequence of a different secreted protein such as IL-1-beta. A signal sequence derived from a protein that is normally secreted from cardiac myocytes can be used. Angiogenic genes can also provide additional functions that can improve, for example cardiac cell function. For example, FGFs can provide cardiac enhancing and/or “ischemic protectant effects” that may be independent of their capability to promote angiogenesis. Thus, angiogenic genes can be used to enhance cardiac function by mechanisms that are additional to or in place of the promotion of angiogenesis per se. As an additional example, IGFs, which can promote angiogenesis, can also enhance muscle cell function (see e.g. Musaro et al. Nature 400: 581-585, 1999); as well as exhibit anti-apoptotic effects (see e.g. Lee et al. Endocrinology 140: 4831-4840, 1999).

Other cardiac genes that can be employed in connection with the present invention include, for example, genes affecting myocardial contractility, promoting cardiac cell survival, recruiting cells to or within the heart, such as cardiac stem cells, or otherwise affecting cardiac physiology. Numerous such genes are known in the art. By way of further illustration, genes that can be used to alter cardiac contractility include, beta-adrenergic signaling proteins (beta-ASPs) (including beta-adrenergic receptors (beta-ARs), G-protein receptor kinase inhibitors (GRK inhibitors) and adenylylcyclases (ACs)) can also be employed to enhance cardiac function as described and illustrated in detail in U.S. patent application Ser. No. 08/924,757, filed 5 Sep. 1997 (based on U.S. 60/048,933 filed 16 Jun. 1997 and U.S. Ser. No. 08/708,661 filed 5 Sep. 1996), as well as PCT/US97/15610 filed 5 Sep. 1997, and U.S. continuing case Ser. No. 09/008,097, filed 16 Jan. 1998, and U.S. continuing case Ser. No. 09/472,667, filed 27 Dec. 1999, each of which is incorporated by reference herein.

As will be apparent to those skilled in the art based on the methods and illustrative teachings of the present invention, other proteins which enhance cardiac function can be employed in accordance with the methods of the present invention.

### Multiple Genes

As noted above, genes encoding one or more angiogenic proteins or peptides can be used in conjunction with the present invention. Thus, a gene or genes encoding a combination of angiogenic proteins or peptides can be delivered using one or more vectors according to the methods described herein. The families of angiogenic genes described herein and in the art comprise numerous examples of such genes. Preferably, where such a combination is employed, the genes may be derived from different families of angiogenic factors (such as a combination selected from two or more different members of the group consisting of FGFs, VEGFs, PDGFs and IGFs). To take a single illustration of such a combination, a vector comprising an FGF gene and a VEGF gene may be used. As an illustrative example, we have used a combination of an FGF gene (FGF-4 fragment 140) (see e.g., the FGF-4 gene and variants thereof described by Basilico et al., in U.S. Pat. No. 5,459,250, issued 17 Oct. 1995, and related cases) and a variant VEGF gene (VEGF-145 mutein 2) (see, e.g., the VEGF-145 gene and variants thereof described by Neufeld et al., WO 98/10071, published 12 Mar. 1998, and related cases). Such combinations can exhibit additive and/or synergistic effects.

Numerous other combinations will be apparent to those of skill in the art based on these teachings. Vectors comprising angiogenic genes or combinations of angiogenic genes, in accordance with the present invention, can also include one or more other genes that can be used to further enhance tissue blood flow and/or contractile function. In the heart, for example, genes encoding beta-ASPs (as described, by Hammond et al., in co-pending applications WO 98/10085, published 12 Mar. 1998) can be employed in combination with one or more genes encoding angiogenic proteins or peptides. Other cardiac or muscle cell enhancing proteins can similarly be incorporated into the compositions and methods of the present invention.

Combinations of genes that can be employed in accordance with the present invention can be provided within a single vector (e.g., as separate genes, each under the control of a promoter, or as a single transcriptional or translational fusion gene). Combinations of genes can also be provided as a combination of vectors (which may be derived from the same or different vectors, such as a combination of adenovirus vectors, or an adenovirus vector and an AAV vector); which can be introduced to a patient coincidentally or in series. In the case of Adenovirus (Ad) and Adeno-associated virus (AAV), the presence of Ad, which is normally a helper virus for AAV, can enhance the ability of AAV to mediate gene transfer. An Ad vector may thus be introduced coincident with or prior to introduction of an AAV vector according to the present invention. In addition to transfection efficiency, the choice of vector is also influenced by the desired longevity of transgene expression. By way of illustration, since many angiogenic genes can bring about long-term effects without requiring long-term expression (e.g., by initiating or facilitating the process of angiogenesis which results in an increase in tissue vascularization), angiogenic genes may be introduced using an adenovirus (or other vector that does not normally integrate into host DNA) which might be used prior to or in combination with the introduction of an AAV vector carrying a transgene for which longer-term expression is desired (e.g., a beta-ASP transgene). Other combinations of transgenes and/or vectors will be apparent to those of skill in the art based on the teachings and illustrations of the present invention.

For treating humans, genes encoding angiogenic proteins of human origin are preferred although angiogenic proteins of other mammalian origin that exhibit cross-species activity i. e. having angiogenic activity in humans can also be used.

### Promoters

In preferred embodiments of the present invention, the nucleic acid is operably linked to a promoter facilitating and potentially controlling expression of one or more genes within the nucleic acid. Selection of the appropriate promoter is based on published data as well as empirical evidence as illustrated herein. Preferably, the gene encoding a protein or peptide employed in the present invention are operably linked to one or more promoters that direct transcription of the gene in a mammalian cell, such as a cell in the heart, skeletal muscle or other target tissue, including for example, a cardiomyocyte, a fibroblast, or an endothelial cell. Presently preferred promoters include generally constitutive promoters such as a cytomegalovirus immediate-early enhancer/promoter (“CMV promoter”), Rous sarcoma virus promoter (“RSV promoter”), Simian Virus 40 (“SV40 promoter”) or human elongation factor-1 alpha/HTLV enhancer (“Hef-1 alpha/HTLV promoter”).

In some embodiments, the gene encoding the protein or peptide is operatively linked to a functional portion of the immediate-early enhancer/promoter regulatory region of human CMV (hCMV). In some embodiments, the gene encoding a protein or peptide is operatively linked to a shortened immediate-early enhancer/promoter regulatory region of hCMV (a truncated CMV promoter or “tCMV promoter”), for example, a promoter of about 517 nucleotides in length, a promoter of about 742 nucleotides in length or a promoter of about 795 nucleotides in length. The sequence of the hCMV promoter regulatory region is known in the art, for example, as described in Bebbington, WO 89/01036 and U.S. Pat. No. 5,168,062 (Stinski) and U.S. Pat. No. 5,385,839 (Stinski). In some embodiments, the gene encoding an angiogenic protein or peptide is operatively linked to an enhanced CMV promoter (“eCMV promoter”) comprising a CMV immediate-early enhancer/promoter and 5′ untranslated region of the major immediate early gene of hCMV. In some embodiments, the eCMV promoter is about 1.7 kb in length.

Other promoter systems include inducible systems (e.g., tetracycline-inducible, ecdysone and others). Alternatively, a tissue-specific promoter, such as a cardiac-specific promoter (e.g., a cardiomyocyte-specific promoter) or skeletal muscle-specific promoter may be employed. Exemplary tissue specific promoters include cardiomyocyte-specific myosin light chain and the cardiomyocyte-specific myosin heavy chain. Many promoters and promoter systems are commercially available through vendors such as Stratagene, Invitrogen Corp., Promega Corp., Invivogen and others. Preferably, the transgene is also operably linked to a polyadenylation signal and may likewise be linked to transcription and/or translation enhancers or similar regulatory sequences. Enhancers exemplified herein are the heat shock protein 70 transcription enhancer (HSP70) and QBI SP163 transcription enhancer (Invitrogen Corp.). An exemplary translation enhancer used herein is the Kozak enhancer element. As will be appreciated by those of skill in the art, post-transcription and/or post-translation signaling sequences may likewise be included in the gene encoding an angiogenic protein or peptide in accordance with the present invention.

### Vectors for Gene Delivery

As stated above, various types of gene delivery vectors are known and readily adaptable for use herein. Vectors useful in the present invention include, for example, plasmids, viral vectors, lipid-based vectors (e.g. liposomes) and the like, capable of delivering a transgene into cells in vitro, ex vivo and/or in vivo thereby facilitating expression of said transgene within said cells. Presently preferred are viral vectors, particularly replication-deficient viral vectors including, for example, replication-deficient adenovirus and adeno-associated virus vectors. For ease of production and use in the present invention, replication-deficient adenovirus vectors are exemplified herein. In contrast to some other viral delivery systems, adenovirus generally does not require host cell replication for gene expression because integration is not normally a component of the adenoviral life cycle. Thus, adenovirus can infect non-dividing cells making it well suited for expressing recombinant genes in nonreplicative cells, such as cardiac myocytes.

A variety of other vectors, both viral and non-viral, can likewise readily be employed to deliver angiogenic protein or peptide encoding nucleic acids in accordance with the present invention. For in vivo use, the vectors are preferably suitable for such by their nature or modifiable for such use. With respect to viral vectors, adenovirus (Ad), adeno-associated virus (AAV), lentivirus (e.g. based on HIV, feline immunodeficiency virus), herpes virus vaccinia virus, various RNA viruses and bovine papillomavirus are exemplary. By way of illustration, AAV vectors useful in the gene therapy methods and compositions of the present invention are preferably replication-deficient in humans, for example, due to deletion of the rep and/or cap genes, essential to AAV replication, and the transgene (including associated promoters and other regulatory sequences) inserted therein is preferably flanked by AAV inverted terminal repeat (ITR) sequences. The resulting recombinant AAV vector is then replicated in a packaging cell line supplying the missing AAV functions (i.e., the rep and/or cap genes) in trans. References describing these and other gene delivery vectors are known in the art, a number of which are cited herein.

Recombinant viral vectors comprise heterologous non-viral genes or sequences. Since many viral vectors exhibit size-constraints associated with packaging, and since replication-deficient viral vectors are generally preferred for in vivo delivery, the heterologous genes or sequences are typically introduced by replacing one or more portions of the viral genome. Such viruses may become replication-deficient as a result of the deletions, thereby requiring the deleted function(s) to be provided in trans during viral replication and encapsidation (by using, e.g., a helper virus or a packaging cell line carrying genes necessary for replication and/or encapsidation) (see, e.g., the references and illustrations of viral vectors herein). Modified viral vectors in which a polynucleotide to be delivered is carried on the outside of the viral particle have also been described (see, e.g., Curiel et al. (1991) Proc. Natl. Acad. Sci. U.S.A. 88:8850-8854).

As described above and in the scientific literature, a number of retrovirus-derived systems have also been developed to be used in gene delivery, particularly in vivo gene delivery. By way of illustration, the lentivirus genus of retroviruses (for example, human immunodeficiency virus, feline immunodeficiency virus and the like) can be modified so that they are able to transduce cells that are typically non-dividing (see, e.g., Naldini et al. (1996) Science 272:263-267; Miyoshi et al. (1998) J. Virol. 72:8150-8157; and Buchschacher et al. (2000) Blood 15:2499-2504; U.S. Pat. No. 6,013,516 (Verma et al.)). While HIV-based lentiviral vector systems have received some degree of focus in this regard, other lentiviral systems have recently been developed, such as feline immunodeficiency virus-based lentivirus vector systems, that offer potential advantages over the HIV-based systems (see e.g., Poeschla et al. (1998) Nat. Med. 4:354-357; Romano et al. (2000) Stem Cells 18:19-39 and references reviewed therein).

In addition to viral vectors, non-viral vectors that may be employed as a gene delivery means are likewise known and continue to be developed. For example, non-viral protein-based delivery platforms, such as macromolecular complexes comprising a DNA binding protein and a carrier or moiety capable of mediating gene delivery, as well as lipid-based vectors (such as liposomes, micelles, lipid-containing emulsions and others) have been described in the art (see e.g., Romano et al. (2000) Stem Cells 18:19-39 and references reviewed therein). Improvements in lipid-mediated in vivo gene delivery have been facilitated by the development of new cationic formulations and vector delivery co-factors (see e.g., Kollen et al. (1999) Hum. Gene Ther. 10:615-622; Roy et al. (1999) Nat. Med. 5:387-391; Fajac et al. (1999) Hum. Gene Ther. 10:395-406; Ochiya et al. (1999) Nat. Med. 5:707-710). Additionally, the development of systems which combine components of viral and non-viral mediated gene delivery systems have been described and may be employed herein (see e.g., Di Nicola et al. (1999) Hum. Gene Ther. 10:1875-1884).

### Targeted Constructs

The present invention contemplates the use of targeting not only by physical means such as delivery of a vector directly into the cardiac muscle, or delivery into a vessel supplying blood thereto or transporting blood therefrom, but also by use of targeted vector constructs having features that tend to target gene delivery and/or gene expression to particular host cells or host cell types (e.g. cardiomyocytes). Such targeted vector constructs would thus include targeted delivery vectors and/or targeted vectors, as described in more detail below and in the published art. Restricting delivery and/or expression is beneficial as a means of further focusing the potential effects of the gene therapeutic and of minimizing any undesirable secondary effects that may be realized by systemic delivery and/or expression. The potential usefulness of further restricting delivery/expression depends in large part on the type of vector being used and the method of introduction of such vector. By way of example, where the vector is delivered to cells ex vivo, further targeting of the vector is not critical. Similarly, as described herein, delivery of viral vectors via intracoronary injection to the myocardium has been observed to provide, in itself, highly targeted gene delivery. However, other means of limiting delivery and/or expression can also be employed, in addition to or in place of the illustrated delivery methods, as described herein.

Targeted delivery vectors include, for example, vectors (such as viruses, non-viral protein-based vectors and lipid-based vectors) having surface components (such as a member of a ligand-receptor pair, the other half of which is found on a host cell to be targeted) or other features that mediate preferential binding and/or gene delivery to particular host cells or host cell types. As is known in the art, a number of vectors of both viral and non-viral origin have inherent properties facilitating such preferential binding and/or have been modified to effect preferential targeting (see, e.g., Douglas et al. (1996) Nat. Biotech. 14:1574-1578; Kasahara et al. (1994) Science 266:1373-1376; Miller et al. (1995) FASEB J. 9:190-199; Chonn et al. (1995) Curr. Opin. in Biotech. 6:698-708; Schofield et al. (1995) British Med. Bull. 51:56-71; Schreier (1994) Pharmaceutica Acta Helvetiae 68:145-159; Ledley (1995) Hum. Gene Ther. 6:1129-1144; Conary et al., WO 95/34647; Overell et al., WO 95/28494; and Truong et al., WO 96/00295).

As stated above and in the cited references, vectors can also comprise components or functionalities that further modulate gene delivery and/or gene expression, or that otherwise provide beneficial properties to the targeted cells. Such other components include, for example, components that influence binding or targeting to cells (including components that mediate cell-type or tissue-specific binding); components that influence uptake of the vector by the cell; components that influence processing and/or localization of the vector and its nucleic acid within the cell after uptake (such as agents mediating intracellular processing and/or nuclear localization); and components that influence expression of the polynucleotide. Such components can also include markers, such as detectable and/or selectable markers that can be used to detect or select for cells that have taken up and are expressing the nucleic acid delivered by the vector. Such components can be provided as a natural feature of the vector (such as the use of certain viral vectors which have components or functionalities mediating binding and uptake), or vectors can be modified to provide such functionalities (such as viruses which have been modified to include a cell binding or targeting protein on the exterior surface of their envelope or capsid). A detectable marker gene allows cells carrying the gene to be specifically detected (e.g., distinguished from cells which do not carry the marker gene). One example of such a detectable marker gene is the lacZ gene, encoding beta-galactosidase, which allows cells transfected with a vector carrying the lacZ gene to be detected by staining, as described below. Other such detectable marker genes include a gene which encodes green fluorescent protein and a gene which encodes a luciferase enzyme, both of which are widely used detectable marker systems. Selectable markers can be positive, negative or bifunctional. Positive selectable markers allow selection for cells carrying the marker, whereas negative selectable markers allow cells carrying the marker to be selectively eliminated. A variety of such marker genes have been described, including bifunctional (i.e. positive/negative) markers (see, e.g., Lupton, WO 92/08796; Lupton, WO 94/28143). Such marker genes can provide an added measure of control that can be advantageous in gene therapy contexts. A large variety of such vectors are known in the art and are generally available (see, e.g., the various references cited above).

References describing adenovirus vectors and other viral vectors which could be used in the compositions and methods of the present invention, in addition to those cited above, include the following: Horwitz, pp. 1679-1721, Adenoviridae and Their Replication, in Fields et al. (eds.) Virology, Vol. 2 (1990) Raven Press New York; Graham et al., pp. 109-128 in Methods in Molecular Biology, Vol. 7: Gene Transfer and Expression Protocols, Murray, E. (ed.), Humana Press, Clifton, N.J. (1991); Miller et al. (1995) FASEB J. 9:190-199; Schreier (1994) Pharmaceutica Acta Helvetiae 68:145-159; Curiel et al. (1992) Hum. Gene Ther. 3:147-154; Graham et al., WO 95/00655; Falck-Pedersen, WO 95/16772; Denefle et al., WO 95/23867; Haddada et al., WO 94/26914; Perricaudet et al., WO 95/02697; Zhang et al., WO 95/25071). A variety of adenovirus plasmids are also available from commercial sources, including, e.g., Microbix Biosystems of Toronto, Ontario (see, e.g., Microbix Product Information Sheet: Plasmids for Adenovirus Vector Construction, 1996).

Additional references describing AAV vectors which could be used in the compositions and methods of the present invention include the following: Carter, Handbook of Parvoviruses, vol. I, pp. 169-228, 1990; Berns, Virology, pp. 1743-1764 (Raven Press 1990); Carter (1992) Curr. Opin. Biotechnol. 3:533-539; Muzyczka (1992) Curr. Top. Microbiol. Immunol. 158:92-129; Flotte et al. (1992) Am. J. Respir. Cell Mol. Biol. 7:349-356; Chatterjee et al. (1995) Ann. NY Acad. Sci. 770:79-90; Kotin (1994) Hum. Gene Ther. 5:793-801; Flotte et al. (1995) Gene Therapy 2:357-362; WO 96/17947; Du et al. (1996) Gene Therapy 3:254-261; Kaplitt et al. (1996) Ann. Thorac. Surg. 62:1669-1676; Samulski et al. (1989) J. Virol. 63:3822-3828; Zolotukhin et al. (1999) Gene Therapy 6:973-985; Atkinson et al., WO 99/11764.

References describing non-viral vectors which could be used in the composition and methods of the present invention include the following: Ledley (1995) Hum. Gene Ther. 6:1129-1144; Miller et al. (1995) FASEB J. 9:190-199; Chonn et al. (1995) Curr. Opin. in Biotech. 6:698-708; Schofield et al. (1995) British Med. Bull. 51:56-71; Brigham et al. (1993) J. Liposome Res. 3:31-49; Philip et al. (1994) Mol. Cell Biol. 14:2411-2418; Perales et al. (1994) Proc. Natl. Acad. Sci. U.S.A. 91:4086-4090; Solodin et al. (1995) Biochemistry 34:13537-13544; Hanson et al., WO 95/25809; Gao et al., WO 96/22765; Brigham, WO 91/06309; Feigner et al., WO 91/17424; Szoka et al., WO 93/19768; Debs et al., WO 93/25673; Overell et al., WO 95/28494; Jessee, WO 95/02698; Haces et al., WO 95/17373; Lin et al., WO 96/01840 and U.S. Pat. No. 5,264,618 (Feigner et al.); U.S. Pat. No. 5,283,185 (Epand et al.); U.S. Pat. No. 5,334,761 (Gebeyehu et al.); U.S. Pat. No. 5,459,127 (Feigner et al.).

The effective dose of the viral vectors of this invention will typically be in the range of about 10E7-10E13 viral particles, preferably about 10E9-10E11 viral particles. As noted, the exact dose to be administered is determined by the attending clinician depending on the desired level of delivery to the myocardium, but preferably, the solution comprising the vector is <10 ml, more preferred is <5 ml of a physiologically buffered solution (such as phosphate buffered saline), still more preferably in 1-3 ml.

### Vasoactive Agents

In a further aspect of the present invention, where the compositions are employed in gene therapy methods, the efficiency of gene delivery using a vector such as a viral vector (e.g. adenovirus or adeno-associated virus) may be enhanced by delivering the vector into a blood vessel or into a tissue that is co-infused or pre-infused with a vasoactive agent. The use of such vasoactive agents has been described and illustrated by e.g., Hammond, WO 99/40945; Hammond, WO 02/089856, each of which are hereby incorporated by reference in their entirety.

Most preferably the vasoactive agent is infused into the blood vessel or tissue coincidently with or within several minutes prior to introduction of the vector composition.

Nitric oxide donors, such as sodium nitroprusside (SNP), can be employed as vasoactive agents. Preferably the nitric oxide donor (e.g., SNP) is pre-infused into the target tissue (or blood vessel supplying a target tissue), beginning several minutes prior to and continuing up until the time of infusion of the vector composition. Administration can also be continued during infusion of the vector composition.

As an illustrative embodiment, a vasoactive agent may be used as described herein to substantially enhance delivery of a vector to an infused site such as the myocardium.

By way of illustration, of other vasoactive agents histamine and other agents may be used as described by Hammond, WO 99/40945. Histamine derivatives and agonists, such as those that interact with histamine H receptors, for example, methylhistamine, 2-pyridylethylamine, betahistine, and 2 thiazolylethylamine can be employed as vasoactive agents. These and additional histamine agonists are described, for example, in Garrison J C., Goodman and Gilman's The Pharmacological Basis of Therapeutics (8th Ed: Gilman A G, Rall T W, Nies A S, Taylor P, eds) Pergamon Press, 1990, pp 575-582 and in other pharmacological treatises.

In addition to histamine and histamine agonists, which can be employed as vasoactive agents, vascular endothelial growth factors (VEGFs) and VEGF agonists (as described herein and in the cited references) can also induce increased vascular permeability and can therefore be used as a vasoactive agent to enhance gene delivery in the context of the compositions and methods described herein. As with histamine, the VEGF is preferably infused into a blood vessel supplying the target site over several minutes prior to infusion of vector.

### Method of Administration

The preferred method of administration of a vector comprising a nucleic acid to the myocardium of a patient in the present invention is by the steps of: (a) inflating a balloon catheter within a coronary artery supplying blood to the myocardium to cause a first occlusion of myocardial blood flow, (b) allowing a first reperfusion of the myocardium by deflating the balloon catheter to release the first occlusion, (c) reinflating the balloon catheter to cause a second occlusion of myocardial blood flow, and (d) infusing a solution comprising the vector into the coronary artery downstream of the site of the second occlusion, and (e) allowing a second reperfusion of the myocardium by deflating the balloon catheter to release the second occlusion.

In an exemplary embodiment of the invention, the above steps of administration are applied in two coronary arteries of the patient. In a more preferred embodiment of the invention, the coronary arteries of the patient are the left descending artery and the left circumflex artery.

In a preferred embodiment, the first occlusion should be at least one minute to several minutes. In a more preferred embodiment, the first occlusion is for approximately three minutes. In a most preferred embodiment of the invention, the patient may be monitored by EKG or other means and observed for indicators of acute ischemia. Upon observance of an indicator of acute ischemia, the occlusion is preferably released as soon as possible. Indicators of acute ischemia are known in the art and include, chest pain, a drop in blood pressure, ST segment depression, and tachycardia. Signs of acute ischemia are an indicator that ischemia has been achieved sufficient to accomplish the goal of increased delivery of a vector comprising a nucleic acid.

In a preferred embodiment, a vasoactive agent is infused into the same artery that was previously occluded after the first occlusion is released. In a more preferred embodiment, the vasoactive agent is infused two minutes after the release of the first occlusion. In a most preferred embodiment, the vasoactive agent is continually infused starting at two minutes after the release of the first occlusion until the completion of the infusion of the solution comprising the vector.

In a preferred embodiment, the vasoactive agent is nitroglycerin which is infused at a rate of 50 micrograms per minute.

In one embodiment of the invention, after the first occlusion is released, the first reperfusion is allowed to continue for at least 3 minutes, more preferably greater than or equal to 5 minutes.

In one embodiment of the invention, infusion of the solution into the occluded artery begins upon the start of the second occlusion. In a preferred embodiment of the invention, infusion of the solution into the occluded artery begins one minute after the start of the second occlusion. In another embodiment, infusion of the solution into the occluded artery begins after the second occlusion is released.

In one embodiment of the invention, the solution comprising the vector is infused into the patient, over a period of at least 20 second, more preferably >30 seconds, still more preferably >60 seconds, more preferably >90 seconds. In preferred embodiment of the invention, the solution comprising the vector is infused into the patient for approximately 30 seconds. In a more preferred embodiment of the invention, the solution comprising the vector is infused into the patient for 60 seconds. In a most preferred embodiment of the invention, the solution comprising the vector is infused into the patient for approximately 90 seconds.

In another preferred embodiment of the invention, the solution comprising the vector is infused at a rate of 2 ml per minute.

### Balloon Catheters

The use of balloon catheters for dilating a blood vessel, e.g. a coronary artery, or other body lumen is well known in the art. Other uses of balloon catheters have been developed as well, for example, temporarily anchoring an instrument within a body lumen so that a surgical or therapeutic procedure can be performed. Patents generally showing various types of balloon catheters and their application include U.S. Pat. No. 4,820,349 (Saab), U.S. Pat. No. 4,681,092 (Cho et al.), U.S. Pat. No. 4,540,404 (Wolvek) and U.S. Pat. No. 4,422,447 (Schifl).

Preferred balloon catheters are made to deliver therapeutic drugs or agents. For example, some balloon catheters deliver a systemic bolus of liquid comprising a drug or vector to a targeted tissue location using an open catheter lumen or channel located at some length along the catheter shaft.

Some balloon catheters include one or more lumens in the balloon such that a bolus of liquid comprising a drug can be delivered to the distal side of the balloon, after the balloon is inflated.

It is also well known in the medical art to employ catheters having shafts formed with a plurality of lumens in instances where it is necessary or desirable to access the distal end of the catheter or a particular internal body location simultaneously through two or more physically separate passageways.

The use of multi-lumen catheters with a balloon is also known in the art, as shown in U.S. Pat. No. 4,869,263 (Segal et al.) and U.S. Pat. No. 4,299,226 (Banka). The Banka patent describes a catheter with a coaxial shaft comprising an inner lumen and outer lumen wherein the inner lumen is able to receive a guide wire or stylet and the outer lumen is able to receive saline solution to inflate a balloon.

The use of balloon catheters in combination with guide catheters to facilitate the positioning of a balloon catheter inside a body lumen is also well known in the art. In one method, the balloon catheter can be placed inside the guide catheter before the guide catheter is inserted into the body lumen, e.g., the blood vessel. In this method, after the distal end of the guide catheter is placed near the intended treatment site, the balloon catheter is moved out of the distal end of the guide catheter and positioned at the intended treatment site.

In a preferred embodiment of the present invention, a guide catheter may be used to position a balloon in a coronary artery. After the balloon is positioned, the balloon may then be inflated to occlude the artery. In a more preferred embodiment, the balloon comprises at least one lumen such that the catheter passes through the balloon so that the solution comprising the vector may be delivered to the distal side of the balloon after the balloon is inflated. The catheter and the balloon surrounding the catheter, when the balloon is inflated, serve to occlude the artery such that no blood can flow past the inflated balloon.

In a preferred embodiment of the invention, a vasoactive agent is also infused. This may be done through the same lumen of a single balloon catheter as described above or a multilumen catheter may be used such that the vasoactive agent and the solution comprising a vector can be separately (but potentially simultaneously) infused to the distal side of the balloon.

### Kits

Compositions or products of the invention may conveniently be provided in the form of formulations suitable for administration to a patient, into the blood stream (e.g. by intra-arterial injection or as a bolus infusion into tissue such as the skeletal muscle). A suitable administration format may best be determined by a medical practitioner. Suitable pharmaceutically acceptable carriers and their formulation are described in standard formulations treatises, e.g., Remington's Pharmaceuticals Sciences by E. W. Martin. See also Wang, Y. J. and Hanson, M. A., “Parental Formulations of Proteins and Peptides: Stability and Stabilizers”, journals of Parental Sciences and Technology, Technical Report No. 10, Supp. 42: 2S (1988).

Vectors of the present invention should preferably be formulated in solution at neutral pH, for example, about pH 6.5 to about pH 8.5, more preferably from about pH 7 to 8, with an excipient to bring the solution to about isotonicity, for example, 4.5% mannitol or 0.9% sodium chloride, pH buffered with art-known buffer solutions, such as sodium phosphate, that are generally regarded as safe, together with an accepted preservative such as metacresol 0.1% to 0.75%, more preferably from 0.15% to 0.4% metacresol. The desired isotonicity may be accomplished using sodium chloride or other pharmaceutically acceptable agents such as dextrose, boric acid, sodium tartrate, propylene glycol, polyols (such as mannitol and sorbitol), or other inorganic or organic solutes. Sodium chloride is preferred particularly for buffers containing sodium ions. If desired, solutions of the above compositions also can be prepared to enhance shelf life and stability. The therapeutically useful compositions of the invention are prepared by mixing the ingredients following generally accepted procedures. For example, the selected components may be mixed to produce a concentrated mixture which may then be adjusted to the final concentration and viscosity by the addition of water and/or a buffer to control pH or an additional solute to control tonicity.

For use by the physician, the compositions will be provided in dosage form containing an amount of a vector of the invention which will be effective in one or multiple doses in order to provide a therapeutic effect. As will be recognized by those in the field, an effective amount of therapeutic agent will vary with many factors including the age and weight of the patient, the patient's physical condition, and the level of angiogenesis and/or other effect to be obtained, and other factors.

The effective dose of the viral vectors of this invention will typically be in the range of about 10E7-10E13 viral particles, preferably about 10E9-10E11 viral particles. Preferably, the solution comprising the vector is <10 ml, more preferred is <5 ml of a physiologically buffered solution (such as phosphate buffered saline), still more preferably in 1-3 ml.

### Animal Models

An important prerequisites for successful studies of cardiovascular gene therapy is the constitution of animal models that are applicable to humans and able to provide useful data regarding the efficacy of gene transfer. Thus, we have made use of porcine models that fulfill these prerequisites. The pig is a particularly suitable model for studying heart diseases of humans because of its relevance to human physiology. The pig heart closely resembles the human heart in the following ways. The pig has a native coronary circulation very similar to that of humans, including the relative lack of native coronary collateral vessels. Secondly, the size of the pig heart, as a percentage of total body weight, is similar to that of the human heart.

Additionally, the pig is a large animal model, therefore allowing more accurate extrapolation of various parameters such as effective vector dosages, toxicity, etc. In contrast, the hearts of animals such as dogs and members of the murine family have a lot of endogenous collateral vessels. Additionally, relative to total body weight, the size of the dog heart is twice that of the human heart.

Finally, the pig model provided an excellent means to follow regional blood flow and function before and after gene delivery. The pig has a native coronary circulation very similar of that of humans, including the relative lack of native coronary collateral vessels. Based on the foregoing, and previous published studies, those skilled in the art will appreciate that the results described below in pigs are expected to be predictive of results in humans.

Thus, these models can be used to determine whether delivery of a vector construct coding for an angiogenic peptide or protein is effective to alleviate the cardiac dysfunctions associated with these conditions. These models are particularly useful in providing some of the parameters by which to assess the effectiveness of in vivo gene therapy for the treatment of congestive heart failure and ventricular remodeling.

### Illustrative Embodiments

The following illustrative embodiments are provided to assist those of ordinary skill in the art and are intended to be illustrative but not limiting with respect to the present invention. Additional variations will become apparent to those of skill in the art and are considered to fall within the scope of the invention as described and claimed herein.

1. A method of delivering a vector comprising a nucleic acid to the myocardium of a patient, comprising the steps of: (a) inflating a balloon catheter within a coronary artery supplying blood to the myocardium to cause a first occlusion of myocardial blood flow, (b) allowing a first reperfusion of the myocardium by deflating the balloon catheter to release the first occlusion, (c) reinflating the balloon catheter to cause a second occlusion of myocardial blood flow, and (d) infusing a solution comprising the vector into the coronary artery downstream of the site of the second occlusion, and (e) allowing a second reperfusion of the myocardium by deflating the balloon catheter to release the second occlusion.

2. A method of embodiment 1, wherein said steps are repeated in a second coronary artery of the patient.

3. A method of embodiment 1 or 2, wherein said step of infusing a solution comprising the vector into the coronary artery is performed coincident with the second occlusion by infusing the solution through an infusion port of the balloon catheter.

4. A method of embodiment 1 or 2, wherein said step of infusing a solution comprising the vector into the coronary artery is performed coincident with the second reperfusion of the myocardium by infusing the solution into the myocardial blood flow after release of the second occlusion.

5. A method according to any one of embodiments 1 to 4, wherein the first occlusion is between one and five minutes in duration.

6. A method of embodiment 5, wherein the first occlusion is between two and three minutes.

7. A method according to any one of embodiments 1 to 6, wherein the first reperfusion is between one and ten minutes in duration.

8. A method of embodiment 7, wherein the first reperfusion is approximately five minutes.

9. A method according to any one of embodiments 1 to 8, wherein the second occlusion is between one and five minutes in duration.

10. A method of embodiment 9, wherein the second occlusion is approximately three minutes.

11. A method according to any one of embodiments 1 to 10, further comprising the step of infusing a vasoactive agent.

12. A method of embodiment 11, wherein the vasoactive agent is infused during a portion of the first reperfusion.

13. A method of embodiment 12, wherein the vasoactive agent is infused during a portion of the first reperfusion and during a portion of the second occlusion by infusing the solution through an infusion port of the balloon catheter.

14. A method according to any one of embodiments 11 to 13, wherein the vasoactive agent is selected from the group consisting of histamine, a histamine agonist, sodium nitroprusside (SNP), an SNP agonist or a vascular endothelial growth factor.

15. A method of embodiment 14, wherein the vasoactive agent is nitroglycerin.

16. A method according to any one of embodiments 1 to 15, wherein the first occlusion is approximately three minutes and the first reperfusion is approximately five minutes.

17. A method according to any one of embodiments 11 to 15, wherein a vasoactive agent is infused beginning approximately two minutes after initiation of the second occlusion and is continued throughout the second occlusion.

18. A method according to any one of embodiments 1 to 17, wherein the solution comprising the vector is infused during the second occlusion.

19. A method of embodiment 18, wherein the solution comprising the vector is slowly infused over a period of at least thirty seconds during the second occlusion.

20. A method of embodiment 19, wherein the solution comprising the vector is slowly infused over approximately ninety seconds during the second occlusion.

21. A method according to any one of embodiments 1 to 20, wherein the vector is a viral vector.

22. A method of embodiment 21, wherein the viral vector is a replication-deficient adenovirus.

23. A method of embodiment 22, wherein the solution comprising the vector contains between about 10E7 to about 10E13 adenovirus vector particles.

24. A method of embodiment 23, wherein the solution comprising the vector contains between about 10E9 to about 10E11 adenovirus vector particles.

25. A method according to any one of embodiments 1 to 24, wherein the nucleic acid encodes a protein.

26. A method of embodiment 25, wherein the protein is selected from the group consisting of a factor which induces angiogenesis, a factor which promotes myocardial contractility, a factor which promotes cardiac cell survival, and a factor which recruits cells within or to the heart.

27. A method of embodiment 26, wherein the protein in an angiogenic protein.

28. A method of embodiment 26, wherein the protein is a growth factor.

29. A method of embodiment 28, wherein the protein is selected from the group consisting of a fibroblast growth factor, a vascular endothelial growth factor, a platelet-derived growth factor, a hypoxia-inducible factor, an angiogenic polypeptide regulator, and an insulin-like growth factor.

30. A method of embodiment 29, wherein the protein is human fibroblast growth factor (hFGF).

31. A method of embodiment 30, wherein the protein is hFGF type IV.

32. A method according to any one of embodiments 1 to 31, wherein the nucleic acid is operably linked to a heterologous promoter.

33. A method of embodiment 32, wherein the heterologous promoter is a constitutive promoter.

34. A method of embodiment 33, wherein the heterologous promoter is a cytomegalovirus (CMV) promoter.

35. A method according to any one of embodiments 25 to 31, wherein the protein is a zinc finger DNA binding protein.

36. A method according to any one of embodiments 25 to 35, wherein the protein is expressed in the heart.

37. A method of embodiment 36, wherein expression of the protein in the heart results in angiogenesis, increased myocardial contractility, promotion of cardiac cell survival, or recruitment of cells within or to the heart.

38. A method of embodiment 37, wherein expression of the protein in the heart results in an increase of myocardial perfusion.

39. A method according to any one of embodiments 25 to 38, wherein the nucleic acid encodes a second protein.

40. A method of embodiment 39, wherein the second protein is selected from the group consisting of a factor which induces angiogenesis, a factor which promotes myocardial contractility, a factor which promotes cardiac cell survival, and a factor which recruits cells within or to the heart.

41. A method according to any one of embodiments 1 to 40, wherein the patient has atherosclerosis.

42. A method according to any one of embodiments 1 to 40, wherein the patient has one or more myocardial perfusion deficits when the heart is imaged under stress.

43. A method according to any one of embodiments 1 to 40, wherein the patient has myocardial ischemia.

44. A method according to any one of embodiments 1 to 40, wherein the patient has experienced angina pectoris.

45. A method according to any one of embodiments 1 to 40, wherein the patient has congestive heart failure.

46. Use of a vector comprising a nucleic acid in the preparation of a medicament for the treatment of heart disease, wherein the medicament is for infusion into the myocardium through introduction into a coronary artery following induced occlusion.

47. Use of a vector comprising a nucleic acid in the preparation of a medicament for the treatment of heart disease, wherein the medicament is for infusion into the myocardium through introduction into a coronary artery following induced occlusion, wherein the infusion into the myocardium is according to a method of any one of embodiments 1 to 45.

48. A combination comprising a medicament for the treatment of heart disease, wherein the medicament is for infusion into the myocardium through introduction into a coronary artery following induced occlusion, and a device for the occlusion of a coronary artery.

49. A combination of embodiment 48, wherein the device is a balloon catheter.

50. A combination of embodiment 49, wherein the balloon catheter comprises an infusion port through which a solution may be infused while the balloon in inflated.

51. A combination according to any one of embodiments 48 to 50, wherein the induced occlusion comprises a first and second occlusion of the coronary artery separated by a first reperfusion.

52. A combination according to any one of embodiments 48 to 51, wherein the induced occlusion comprises the steps of: (a) inflating a balloon catheter within a coronary artery supplying blood to the myocardium to cause a first occlusion of myocardial blood flow, (b) allowing a first reperfusion of the myocardium by deflating the balloon catheter to release the first occlusion, (c) reinflating the balloon catheter to cause a second occlusion of myocardial blood flow, and (d) infusing a solution comprising the vector into the coronary artery downstream of the site of the second occlusion, and (e) allowing a second reperfusion of the myocardium by deflating the balloon catheter to release the second occlusion.

53. A combination comprising a medicament for the treatment of heart disease, wherein the medicament is for infusion into the myocardium through introduction into a coronary artery following induced occlusion, and a device for the occlusion of a coronary artery, wherein the induced occlusion is performed according to any one of embodiments 1 to 45.

### EXAMPLES

Based on these results, some of which are described in detail in the Examples below, it is seen that a significant degree of targeted in vivo gene transfer is achieved.

The following Examples are provided to further assist those of ordinary skill in the art.

Such examples are intended to be illustrative and therefore should not be regarded as limiting the invention. A number of exemplary modifications and variations are described in this application and others will become apparent to those of skill in this art. Such variations are considered to fall within the scope of the invention as described and claimed herein.

### Example 1

### Porcine Model of Intermittent Coronary Artery Occlusion

A porcine model of intermittent occlusion was developed to provide an animal model to test the delivery of nucleic acids to the heart that would closely approximate the physiological characteristics of humans. As an illustrative embodiment of the present invention, we used three minutes of occlusion of a coronary artery followed by five minutes of reperfusion followed by a second three minutes of occlusion of the same coronary artery with the dosing of a replication deficient adenovirus comprising a nucleic acid encoding luciferase (Ad5Luc) into the occluded coronary artery during the second occlusion starting at one minute after the second occlusion had begun.

Four pigs (Group A) were fasted for at least 12 hours before surgery. The pigs were sedated using a cocktail of ketamine (22 mg/kg, IM), acepromazine (1.1 mg/kg, IM), and atropine (0.05 mg/kg, IM). Following adequate sedation (about 15 minutes) the pigs were moved to the pre-operative care room. A fentanyl patch (50 micrograms per hour, transdermal) was placed on each pig's back. An intravenous catheter was placed in a marginal ear vein for the administration of pentobarbital (10 mg/kg, IV) to induce anesthesia. An appropriately sized endotracheal tube was inserted for mechanical ventilation. The chest and groin were then shaved and prepped for a catheterization procedure.

The pigs were subsequently each placed on a heating blanket to maintain rectal temperature at 37° C. Cefazolin (25 mg/kg) and aspirin (325 mg) were administered intravenously prior to the experiment. All surgical procedures were carried out using standard sterile techniques including sterile gowns, gloves, masks and instruments. All staff wore standard protective clothing including scrubs, shoe covers and masks while in the room. Once in the catheterization suite, the in-dwelling marginal ear vein angiocath on each pig was connected to a saline drip to maintain adequate hydration (10 ml/kg/hr) throughout the study, and for the administration of drugs as needed. Each pig was ventilated on oxygen enriched room air at initial settings of 10 breaths per minute and 10 ml/kg tidal volume. General anesthesia was maintained on each pig with isoflurane (1-2% with oxygen) through the endotracheal tube. Ventilatory rate was adjusted to maintain pCO2 between 30-40 mmHg; pO2 greater than 100 mmHg and pH between 7.35-7.45. If unable to counteract acidemia by changing respiratory settings, acidemia was treated with sodium bicarbonate (2-10 ml; 8.4% solution). Arterial blood gases were taken routinely to ensure physiological stability. Surface EKG leads were placed to provide continuous electrocardiographic data. The chest and groin of each pig were scrubbed with Nolvasan Scrub followed by alcohol-soaked gauze in a series of 3 cycles. EKG electrodes were placed subdermally to monitor the cardiac rhythm of each pig. Betadine solution was sprayed on the surgical sites. Each pig was draped in the usual fashion and sterile instruments were handed off to the surgeon.

In the left descending artery (LAD) coronary artery of two pigs, an 8F sheath was inserted through a left femoral artery cut-down, and a guide catheter was introduced. Likewise, in the left circumflex (LCx) coronary artery of two additional pigs, an 8F sheath and guide catheter were inserted following the same procedures as described above. In each of the four pigs, an angiogram was obtained to visualize the coronary architecture. Following instrumentation, amiodarone (8 mg/kg, I.V.) was given to reduce arrhythmias. A guide catheter was positioned at the left main coronary artery ostium, and a 0.014 wire with an over-the-wire double lumen catheter was manipulated into the LAD or LCx coronary artery and positioned at the first diagonal branch. After placement of the intracoronary catheter, each pig was allowed to stabilize for 10 minutes. Baseline hemodynamic measurements (heart rate, blood pressure, EKG) were recorded. An angioplasty balloon was then advanced to below the first or second diagonal branch and inflated to totally occlude the LAD (or LCx) coronary artery. After three minutes of occlusion, the angioplasty balloon was deflated and reperfusion allowed for five minutes. Starting at five minutes of reperfusion the balloon was re-inflated to occlude the artery for a second time.

A frozen test article containing 1×10E11 viral particles of a replication deficient adenovirus comprising a transgene encoding a luciferase as a marker (Ad5Luc) was thawed. Using sterile procedures, the test aliquot was aspirated into a 15 mL syringe through a plastic needle and diluted with normal saline to a volume of 10 mL. The syringe with diluted test article was gently inverted several times to thoroughly mix the contents. The syringe was mounted on the side-port via a wide-bore stopcock.

After one minute of the second occlusion, the prepared 10 mL solution of Ad5Luc was infused into the occluded artery via the second lumen of the intracoronary catheter at a rate of 10 mL/min using a Harvard Apparatus syringe pump. In each pig, when virus infusion was completed, the catheter system was flushed with 2 mL of saline. Following injection and flush, the placement of the intracoronary catheter was confirmed again by cineangiogram. In each of the pigs, after three minutes of the second occlusion, the occlusion was released. The catheters were withdrawn, and the arterial cut-downs were ligated and the overlying tissue was closed in layers. Each pig was transported to the post-operative room and returned to quarantine quarters for 24 hours after being ambulatory and stable. The pigs were returned to general housing after 24 hours. The pigs were observed for 3 days. On day 3, the each pig was premedicated, intubated and anesthetized; the chest was opened by median sternotomy, and the heart was excised. Samples were then taken of the ischemic tissue from the occluded artery. Samples were also taken from the remote non-ischemic right ventricular myocardium as a control. All tissue samples were fresh frozen in liquid nitrogen and analyzed for luciferase expression.

In sample tissue taken from the ischemic areas from the occluded arteries of the Group A pigs, an average of 35.88 picograms of luciferase per gram of tissue were found. In sample tissue taken from the remote non-occluded right ventricular myocardium of those same Group A pigs, no measurable amount of luciferase was found.

In contrast to the four pigs in Group A described above, using the same procedures, four additional pigs (Group B) were injected with an Ad5Luc solution containing 1×10E11 viral particles following a single 3 minute occlusion. The Group B pigs were infused with 5 ml of Ad5Luc solution at 2 mL/min in the LAD or LCx, starting either immediately upon release of a three minute occlusion or at eight minutes after the release of a three minute occlusion. In sample tissue taken from the ischemic tissue (tissue near the occlusion) of the four Group B pigs, very low levels (ranging from 0 to 2 picograms per gram of tissue) of luciferase were found.

Much higher levels of expression were observed in the Group A pigs (averaging on the order of 36 picograms per gram of tissue) representing an unexpected dramatic improvement in gene transfer and expression when the nucleic acid was delivered to the heart using multiple occlusions as described herein.

Genomic DNA was extracted from myocardial tissues of the Group A pigs three days after the initial occlusion/reperfusion, both from ischemic tissue of the occluded artery and from the remote non-ischemic regions of the myocardium. Primers were designed targeting the Ad5Luc transgene. PCR was performed. 10 microliters of PCR products as well as 5 microliters of PCR marker containing 30-40 ng of each DNA fragment were separated by electrophoresis on a 2% agarose gel and visualized with ethidium bromide under UV fluorescence. DNA extracted from the non-ischemic region did not display a signal that would indicate the presence of the Ad5Luc while DNA extracted from the ischemic region showed a positive signal demonstrating the presence of Ad5Luc.

### Example 2

### Porcine Model of Intermittent Coronary Artery Occlusion with a Vasoactivator Agent

As a means of further improving the delivery of nucleic acids to a heart experiencing intermittent occlusion, a vasoactive agent was further incorporated into a porcine model. In the illustrative embodiment, the model employed 3 minutes of coronary artery occlusion followed by five minutes of reperfusion followed by a second 3 minutes of occlusion of the same coronary artery with dosing of a replication deficient adenovirus comprising a nucleic acid encoding luciferase (Ad5Luc) into the occluded artery during the second occlusion starting at one minute after the second occlusion had begun. Further, starting at the second minute of reperfusion an infusion of nitroglycerin was begun and was continued until completion of the Ad5Luc infusion.

Pigs were prepared as described above with respect to Group A. Starting at two minutes of reperfusion, nitroglycerine was infused into a coronary artery of the pigs at a rate of 50 micrograms per minute and continued until completion of the Ad5Luc infusion. After one minute of the second occlusion, the prepared 10 mL solution of Ad5Luc was infused into the LAD via the second lumen of the intracoronary catheter at a rate of 10 mL/min using a Harvard Apparatus syringe pump. When the virus and nitroglycerin infusion was completed, the catheter system was flushed with 2 mL of saline. Following injection and flush, the placement of the intracoronary catheter was confirmed again by cineangiogram. In each of the pigs, after three minutes of the second occlusion, the occlusion was released. The catheters were withdrawn, and the arterial cut-downs were ligated and the overlying tissue was closed in layers. Each pig was transported to the post-operative room and returned to quarantine quarters for 24 hours after being ambulatory and stable. The pigs were returned to general housing after 24 hours. The pigs were observed for 3 days. On day 3, the each pig was premedicated, intubated and anesthetized; the chest was opened by median sternotomy, and the heart was excised. Samples were then taken of the ischemic tissue from the occluded artery. Samples were also taken from the remote non-ischemic right ventricular myocardium as a control. All tissue samples were fresh frozen in liquid nitrogen and analyzed for luciferase expression.

In sample tissue taken from the ischemic areas from the occluded arteries an average of 82.27 picograms of luciferase per gram of tissue were found. In sample tissue taken from the remote non-occluded right ventricular myocardium of those same pigs, no measurable amount of luciferase was found. Genomic DNA was extracted from ischemic and non-ischemic areas of the myocardium of the pigs and subjected to PCR analysis as described above with respect to the Group A pigs. Consistent with the luciferase data, no PCR signal from Ad5Luc was detected in the DNA extracted from non-ischemic area of the myocardium. In DNA extracted from the ischemic region of the myocardium a positive signal from Ad5luc was detected three days after the occlusion/reperfusion procedure which appeared to further increase after nitroglycerin co-administration.

### Example 3

### Illustrative Infusion Procedure for Human Subjects Using an Angiogenic Adenovector (Ad5-FGF4)

Following for purposes of illustration and not limitation, is an exemplary infusion protocol embodying and applying aspects of the present invention (including induced occlusion) to the treatment of patients with myocardial ischemia due to coronary artery disease to be treated with an adenoviral vector containing a nucleic acid encoding human fibroblast growth factor 4 (Ad5-FGF4)—as an illustrative product to be administered—using an exemplary protocol that includes the use of a vasoactive agent (in this example, nitroglycerin).

Patients receive product infusion using a coronary balloon angioplasty catheter. Use of heparin or other anticoagulant is recommended. A single or double lumen catheter is recommended. If a single lumen over-the-wire catheter is used, the wire is removed for product infusion and flush. If a rapid exchange balloon catheter is used, one with a distal infusion lumen/port is preferably used. If a double lumen is used, the wire does not need to be removed during infusion in the second lumen. With a double lumen cather, one lumen may be used for the wire which can be left in place to stabilize the catheter and the other lumen used for infusion distal to the inflated balloon.

An exemplary infusion protocol for induced occlusion is as follows:

(1) A balloon angioplasty catheter is placed in the proximal coronary artery at a site without significant stenosis for balloon inflation.
(2) The balloon is inflated to about one atmosphere or just sufficient to occlude the artery for two minutes as tolerated and deemed safe by the investigator or until evidence of moderate ischemia (such as moderate chest pain, ventricular arrythmias, significant blood pressure change, or additional 1 mm flat/downsloping ST depression). The balloon is preferably inflated to a pressure sufficient to occlude flow, less than would be used for angioplasty or stent deployment, to avoid damage to the endothelium or intima of the artery. Clinical judgment is used to determine if ischemia requires balloon deflation.
(3) Five minutes are allowed for recovery (i.e. reperfusion). After the first 2 minutes of recovery infuse in the same coronary artery (through lumen distal to the balloon) approximately 200 micrograms of nitroglycerin (“NTG”), as an illustrative vasoactive agent, slowly (generally over about 10-30 sec, depending on volume).
(4) There is a second balloon inflation for product infusion and flush, up to 3 minutes as tolerated, again at a pressure just sufficient to occlude flow.
(5) Product (for this example Ad5-FGF4, 6.0×10E9 particles in 5 ml) is infused during the second balloon inflation through distal lumen at approximately 1.5 ml/min followed by saline flush sufficient to clear the dead space in the catheter tubing system at 1.5 ml/min. The balloon should remain inflated for the entire three minutes, as tolerated.
(6) Repeat the procedure for each major coronary artery or graft.

If moderate ischemia requires balloon deflation before product infusion and flush are complete, continue product infusion and flush to completion with the balloon deflated. In monitoring for ischemia to determine if balloon deflation is needed for patient safety, it is recommended that at least 3 EKG leads be used, two limb leads and one precordial lead, that blood pressure is continually monitored, and that the patient be instructed to report the severity of any pain. Depending on clinical judgment, patients may remain hospitalized and be monitored (e.g. for a period of six hours after product administration) and then be discharged to home according to standard practice of the institution.

If either the LCx or LAD is totally occluded (and the RCA is patent) without patent bypass grafts, patients may not tolerate occlusion of the open branch of the left coronary artery. In that case, subselection of a branch of the patent artery may be used.

For Ad5-FGF4 infusion, the catheter position should preferably not be distal to any major branch of the coronary artery supplying that region of the heart. The exception is an infusion into an Internal Mammary Artery (IMA) graft. In this situation care should be taken that the end-hole of the catheter is distal to the branches supplying the thorax. Whenever the catheter is removed, the sheath is preferably flushed with heparinized saline. In general, sub-selective catheter positions that engender a significant risk of complication should not be used.

The location of the balloon used to occlude the coronary artery of the bypass graft is preferably in an area free of stenosis; and the balloon is inflated only sufficient to occlude flow (less than used for angioplasty) and avoid damage to the intima. Each dose of product and flush is preferably infused at a slow, constant rate of approximately 1.5 mL/min by the investigator using a clock as a guide. The total volume for the sub-selective infusion will include the dose of study product followed by sufficient volume of saline to clear the catheter tubing system of study product (the dead space in the system). This volume should be determined prior to administration.

If Ad5-FGF4 is infused through the guide wire lumen of an over-the-wire balloon catheter then each Ad5-FGF4 infusion is preferably immediately followed by an infusion of normal saline to flush the complete dose of product through the catheter/connector tubing dead space. It is preferable to administer the saline flush at the specified infusion rate of approximately 1.5 ml/min. A saline flush volume of 2.0 mL should be used if the length of connecting tubing used to connect the syringe to the catheter is 60 cm. If the connecting tubing is >60 cm in length the saline flush volume is preferably increased to ensure that Ad5-FGF4 entire dose is flushed from the tubing/catheter dead space into the target coronary artery.

1. A method of delivering a vector comprising a nucleic acid to the myocardium of a patient, comprising the steps of: (a) inflating a balloon catheter within a coronary artery supplying blood to the myocardium to cause a first occlusion of myocardial blood flow, (b) allowing a first reperfusion of the myocardium by deflating the balloon catheter to release the first occlusion, (c) re-inflating the balloon catheter to cause a second occlusion of myocardial blood flow, and (d) infusing a solution comprising the vector into the coronary artery downstream of the site of the second occlusion, and (e) allowing a second reperfusion of the myocardium by deflating the balloon catheter to release the second occlusion.

2. A method of claim 1, wherein said step of infusing a solution comprising the vector into the coronary artery is performed coincident with the second occlusion by infusing the solution through an infusion port of the balloon catheter.

3. A method according to claim 1, wherein the first occlusion is between one and five minutes in duration.

4. A method of claim 3, wherein the first occlusion is approximately three minutes.

5. A method of claim 1, wherein the first reperfusion is approximately five minutes.

6. A method of claim 1, wherein the second occlusion is approximately three minutes.

7. A method of claim 1, further comprising the step of infusing a vasoactive agent.

8. A method of claim 7, wherein the vasoactive agent is infused during a portion of the first reperfusion and during a portion of the second occlusion by infusing a solution comprising the vasoactive agent through an infusion port of the balloon catheter.

9. A method of claim 8, wherein the vasoactive agent is nitroglycerin.

10. A method of claim 1, wherein the vector is a replication-deficient adenovirus.

11. A method of claim 1, wherein the nucleic acid encodes a protein selected from the group consisting of a factor which induces angiogenesis, a factor which promotes myocardial contractility, a factor which promotes cardiac cell survival, and a factor which recruits cells within or to the heart.

12. A method of claim 11, wherein expression of the protein in the heart results in angiogenesis, increased myocardial contractility, promotion of cardiac cell survival, or recruitment of cells within or to the heart.

13. A method of claim 12, wherein expression of the protein in the heart results in an increase of myocardial perfusion.

14. A method according to claim 1, wherein the patient has myocardial ischemia.

15. A combination comprising a medicament for the treatment of heart disease, wherein the medicament is for infusion into the myocardium through introduction into a coronary artery following induced occlusion, and a device for the occlusion of a coronary artery.

16. A combination of claim 15, wherein the device is a balloon catheter.

17. A combination of claim 16, wherein the balloon catheter comprises an infusion port through which a solution may be infused while the balloon in inflated.

18. A combination of claim 15, wherein the induced-occlusion comprises a first and second occlusion of the coronary artery separated by a first reperfusion.

19. A combination of claim 15, wherein the induced occlusion comprises the steps of: (a) inflating a balloon catheter within a coronary artery supplying blood to the myocardium to cause a first occlusion of myocardial blood flow, (b) allowing a first reperfusion of the myocardium by deflating the balloon catheter to release the first occlusion, (c) reinflating the balloon catheter to cause a second occlusion of myocardial blood flow, and (d) infusing a solution comprising the vector into the coronary artery downstream of the site of the second occlusion, and (e) allowing a second reperfusion of the myocardium by deflating the balloon catheter to release the second occlusion.
